(19) United States
(12) Patent Application Publication
(10) Pub. No.: US 2010/0286164 A1
(43) Pub. Date: Nov. 11, 2010

(54) SUBSTITUTED ARYL ALKYLAMINO-OXY-ANALOGS AND USES THEREOF

(75) Inventors: Corinne Elizabeth Augelli-Szafran, Newton, MA (US); Michael S. Wolfe, Newton, MA (US); Han-Xun Wei, Somerville, MA (US)

Correspondence Address: WOLF GREENFIELD & SACKS, P.C., 600 ATLANTIC AVENUE, BOSTON, MA, 02210-2206, US

(73) Assignee: The Brigham and Women's Hospital, Inc, Boston, MA (US)

(21) Appl. No.: 12/682,660

(22) PCT Filed: Oct. 10, 2008
(86) PCT No.: PCT/US08/11649

§ 371 Date: Jul. 27, 2010

Related U.S. Application Data
(60) Provisional application No. 60/998,767, filed on Oct. 12, 2007.

(51) Int. Cl. A61K 31/496; C12N 9/99; A61K 31/395; A61K 31/495; A61K 31/438; A61K 31/44; A61K 31/357; A61K 31/135; C07D 225/02; C07D 401/12; C07D 241/04; C07D 491/113; C07D 213/36; C07D 319/16; C07C 215/20; A61P 25/28

(52) U.S. Cl. 514/253.06; 435/184; 514/183; 514/255.03; 514/278; 514/357; 514/452; 514/653; 540/450; 544/363; 544/394; 546/19; 546/334; 549/362; 564/355

(57) ABSTRACT

Aspects of the invention relate to substituted aryl propylamino-oxy-analogs and uses thereof. Aspects of the invention relate to compositions that are inhibitors of γ-secretase and uses thereof for treating subjects having, or at risk of developing, Alzheimer's disease.

### RELATED APPLICATIONS

This application claims the benefit under 35 U.S.C. §119(e) of U.S. provisional patent application Ser. No. 60/998,767, filed Oct. 12, 2007, the contents of which are incorporated herein by reference in their entirety.

### FIELD OF THE INVENTION

Methods and compositions of the invention relate to inhibitors of γ-secretase and uses thereof.

### BACKGROUND OF THE INVENTION

Accumulating biochemical, histological, and genetic evidence supports the hypothesis that the 4 kDa β-amyloid protein (Aβ) is an essential component in the pathogenesis of Alzheimer's disease (AD). Selkoe D J, Science 275:630-631 (1997). Hardy J, Proc Natl Acad Sci USA 94:2095-2097 (1997). Despite the intense interest in the role of Aβ in the etiology of AD, the molecular mechanism of Aβ biosynthesis is poorly understood. The 39-43-residue Aβ is formed via the sequential cleavage of the integral membrane amyloid precursor protein (APP) by β- and γ-secretases. Selkoe D J, Annu Rev Cell Biol 10:373-403 (1994). β-Secretase cleavage of APP occurs near the membrane, producing the soluble APP₈-β and a 12 kDa C-terminal membrane-associated fragment (CTF). The latter is processed by γ-secretase, which cleaves within the transmembrane domain of the substrate to generate A. An alternative proteolytic event carried out by α-secretase occurs within the Aβ portion of APP, releasing APP₈-α, and subsequent processing of the resulting membrane-bound 10 kDa CTF by γ-secretase leads to the formation of a 3 kDa N-terminally truncated version of Aβ called p3.

Heterogeneous proteolysis of the 12 kDa CTF by γ-secretase generates primarily two C-terminal variants of Aβ, 40- and 42-amino acid versions (Aβ₄₀ and Aβ₄₂), and parallel processing of the 10 kDa CTF generates the corresponding C-terminal variants of p3. Although Aβ₄₂ represents only about 10% of secreted Aβ, this longer and more hydrophobic variant is disproportionally present in the amyloid plaques observed post mortem in AD patients (Roher A E et al., Proc Natl Acad Sci USA 90:10836-40 (1993); Iwatsubo T et al., Neuron 13:45-53 (1994)), consistent with in vitro studies illustrating the kinetic insolubility of Aβ₄₂ vis-à-vis Aβ₄₀. Jarrett J T et al., Biochemistry 32:4693-4697 (1993). Importantly, all genetic mutations associated with early-onset (<60 years) familial Alzheimer's disease (FAD) result in increased Aβ₄₂ production. Selkoe D J, Science 275:630-631 (1997); Hardy J, Proc Natl Acad Sci USA 94:2095-2097 (1997). An understanding of the production of Aβ in general and that of Aβ₄₂ in particular is essential for elucidating the molecular mechanism of AD pathogenesis and may also lead to the development of new chemotherapeutic agents which strike at the etiological heart of the disease.

Both γ-secretase and β-secretase are attractive targets for inhibitor design for the purpose of inhibiting production of Aβ. While γ-secretase is an attractive target for inhibitor design, little is known about the structure, mechanism, or binding requirements of this protease. In view of the foregoing, a need still exists to develop compositions and methods for treating disorders characterized by the production and deposition of β-amyloid.

### BRIEF SUMMARY OF THE INVENTION

The invention provides methods, compositions, and articles of manufacture for the treatment of subjects having neurodegenerative disorders, including Alzheimer's disease. These methods include the step of administering to the subject a therapeutically effective amount of γ-secretase inhibitor compound or a therapeutic preparation, composition, or formulation of the compound such as those described herein, including those in the claims. As used herein, it should be appreciated that the term inhibitor refers to a compound that modulates (e.g., reduces) the activity of its target (e.g., protease) regardless of the mode of action of the inhibitor. Accordingly, in some embodiments an inhibitor may react at the active site (e.g., catalytic site) of a protease thereby reducing its activity (e.g., inactivating the protease). In some embodiments, an inhibitor may be a transition state inhibitor. In some embodiments, an inhibitor may be a modulator (e.g., an allosteric modulator) that inhibits protease activity by binding to a modulatory site that indirectly alters the configuration of the active site, substrate binding site, or other site (or combination thereof) thereby modulating the activity of the protease (e.g., reducing the activity of the protease, changing the specificity of the protease, etc., or any combination thereof). In some embodiments, an inhibitor may modulate protease activity either by binding to the protease or to a substrate (or a combination thereof) thereby reducing the activity of the protease for the substrate. In some embodiments, an inhibitor may bind to the protease at a position that interferes with one or more substrate binding and/or product release steps. It should be appreciated that aspects of the invention are not limited by the precise mode of action of the inhibitor and that any direct or indirect effect on the activity of a protease may result from contacting γ-secretase with an inhibitor of the invention. In some embodiments, without wishing to be limited by theory, an inhibitor of the invention may bind to a proposed modulatory site on γ-secretase (see, e.g., Lazarov et. al., P.N.A.S., vol. 103, p. 6889). It also should be appreciated that an inhibitor of the invention may partially or completely inhibit the secretase activity (e.g., by about 10%, about 20%, about 30%, about 40%, about 50%, about 60%, about 70%, about 80%, about 90%, about 95%, or by less or more than any of these values, for example, by 100%, or by any intermediate percentage). In some embodiments, inhibition may be specific (e.g., substrate specific) in that the inhibitory effect is stronger for a first substrate than a second substrate. In some embodiments, specific inhibitors of the invention reduce degradation of the amyloid precursor protein to a greater extent than the Notch protein (e.g., the ratio of % inhibition of amyloid precursor protein degradation to % inhibition of Notch protein degradation is greater than 1). In some embodiments, amyloid precursor protein degradation by γ-secretase may be inhibited by a compound of the invention, whereas Notch degradation by γ-secretase may be unaffected or only slightly inhibited. Certain aspartyl proteases, including γ-secretase, generate β-amyloid from amyloid precursor protein (APP), which may result in neurodegenerative disorders. The γ-secretase inhibitor compounds are useful for treating a subject having or at risk of developing a neurodegenerative disorder associated with γ-secretase activity, e.g., Alzheimer's disease. In some aspects, specific inhibitors of the invention may be used to treat or prevent Alzheimer's disease without causing side effects associated with inhibition of Notch degradation.

Furthermore, some of the compounds selectively inhibit γ-secretase-mediated cleavage of APP with little or no inhibition of the γ-secretase-mediated cleavage of the Notch family of transmembrane receptors. Selective inhibition of the cleavage of APP relative to that of the Notch receptors is believed to minimize certain unwanted side effects, including lymphopoiesis and intestinal cell differentiation.

In some embodiments, the invention provides a method for treating a subject having or at risk of having neurodegenerative disorders, including Alzheimer's disease by administering a γ-secretase inhibitor of the formula:

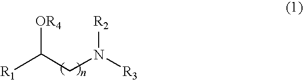

wherein R1 is selected from an aryl ring system; R2 is alkyl or hydrogen; R3 is either an alkyl group further substituted with alkyl or aryl groups or R3 is an aromatic ring system optionally substituted, or when taken together, R2 and R3 form an N,N′-substituted piperazine; and R4 is selected from hydrogen, alkyl, or aryl substituents. In some embodiments, n=1-4. In some embodiments, n=1. In some embodiments, n=2. In some embodiments, n=3. In some embodiments, n=4.

In some embodiments, the invention provides a method for treating a subject having or at risk of having neurodegenerative disorders, including Alzheimer's disease by administering a γ-secretase inhibitor of the formula:

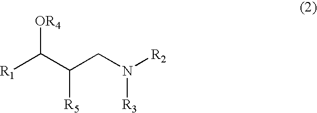

wherein R1 is selected from an aryl ring system; R2 is alkyl or hydrogen; R3 is either an alkyl group further substituted with alkyl or aryl groups or R3 is an aromatic ring system optionally substituted, or when taken together, R2 and R3 form an N,N′-substituted piperazine; R4 is selected from hydrogen, alkyl, and aryl substituents; and R5 is alkyl or hydrogen.

In some embodiments, the invention provides a compound of formula (1), (I), or (2), or a pharmaceutical composition comprising the compound as described herein.

It should be appreciated that a compound of formula (1), (I), or (2) may be used to inhibit γ-secretase activity by contacting γ-secretase (e.g., in vitro or in vivo) with any one or more of the compounds.

The invention also relates to methods of making medicaments for use in treating a subject, e.g., for treating a subject having a condition associated with γ-secretase activity, or at risk of developing a condition associated with γ-secretase activity, treating a subject having Alzheimer's disease, or at risk of developing Alzheimer's disease, inhibiting APP cleavage, and/or inhibiting γ-secretase activity. Accordingly, one or more compounds or compositions described herein that inhibit γ-secretase activity as described herein may be used for the preparation of a medicament for use in any of the methods of treatment described herein. In some embodiments, the invention provides for the use of one or more compounds or compositions of the invention for the manufacture of a medicament or pharmaceutical for treating a mammal (e.g., a human) having one or more symptoms of, or at risk for, a condition associated with γ-secretase activity (e.g., Alzheimer's disease).

### DETAILED DESCRIPTION OF THE INVENTION

Aspects of the invention relate to compositions and methods for treating neurological disorders, including Alzheimer's disease, with certain amino alcohol compounds or derivatives thereof. Aspects of the invention, are based, at least in part, on the discovery that certain amino alcohol derivatives are inhibitors of γ-secretase. In certain embodiments, compositions of the invention are formulated as pharmaceutical compositions. In some embodiments, compositions of the invention are administered to a patient that has, or is at risk of developing, a neurological disorder associated with γ-secretase activity (e.g., abnormally high levels of γ-secretase activity).

In some embodiments, abnormally high levels of γ-secretase activity imply statistically significantly higher levels (e.g., 10% higher, 20% higher, 30% higher, 50% higher, or higher) than a reference level characteristic of normal levels of activity.

However, it should be appreciated that AD patients or those at risk of developing AD may not necessarily have elevated levels of γ-secretase and/or elevated γ-secretase activity. Instead such subjects may suffer the effects of Aβ, which is pathogenic, and which can be produced by γ-secretase at all levels. In some embodiments, elevated levels of Aβ are pathogenic. Levels of Aβ depend on a balance between production and clearance, and there are many factors that are involved in the production and clearance of Aβ. Accordingly, in some embodiments decreasing the γ-secretase-mediated production of Aβ can slow, halt and/or prevent the neurodegenerative effects of Aβ. Therefore, decreasing the γ-secretase production of Aβ, (by up to 10%, or up to 20%, or up to 30%, or up to 40%, or up to 50%, or higher) relative to a baseline activity, can yield a therapeutic effect, and/or prevent disease onset, and/or delay the onset of AD. It should be appreciated that γ-secretase activity in a subject can be measured from Aβ levels in plasma and CSF. Accordingly, levels of Aβ inhibition can be assayed by measuring Aβ levels in the plasma and CSF with different compounds and comparing the levels to a reference level obtained without a test compound or using a compound that is known not to affect Aβ inhibition (e.g., a reference compound that is not a γ-secretase inhibitor).

In some embodiments, compositions of the invention are administered to a patient that has, or is at risk of developing, Alzheimer's disease. In some embodiments, amino alcohol derivatives described herein have the formula:

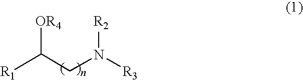

wherein R1 is selected from an aryl ring system; R2 is alkyl or hydrogen; R3 is either an alkyl group further substituted with alkyl or aryl groups or R3 is an aromatic ring system optionally substituted, or when taken together, R2 and R3 form an N,N′-substituted piperazine; and R4 is selected from hydrogen, alkyl, or aryl substituents. Aspects of the invention are based, at least in part, on the discovery that certain reduced ketones, or derivatives thereof, are stable and selective inhibitors of APP cleavage or processing.

In some aspects, compositions of the invention specifically inhibit cleavage of the integral membrane amyloid precursor protein (APP) without significantly inhibiting cleavage of the Notch family of transmembrane receptors. In some embodiments, specific inhibitor compounds have the following general properties: an R1 that is aromatic, an R2 and R3 with at least one substituent that contains an aromatic group substituent and an R4 that is hydrogen or substituted alkyl, phenyl or benzyl substituents (e.g., alkyl, halogen CF3, etc.). In some embodiments, specific inhibitor compounds have a carbon linkage of n=2.

In some embodiments, R1 is a bicyclic carbocyclic ring system or an aromatic ring system. In certain embodiments R1 is phenyl. In some embodiments R1 is substituted phenyl, wherein the substituent is one or more of halogen, methyl, isopropyl, methoxy, and trifluoromethyl. In certain embodiments, R1 is naphthalene. In some embodiments, R1 is a substituted aryl (e.g., naphthalene or phenyl), wherein the substituent may be halogen, and/or methoxy. It should be appreciated that the term naphthalene implies the naphthyl substituent where appropriate, as used herein in the context of a compound of the formula (1) and/or (I) and/or (2).

In some embodiments R4 is hydrogen. In certain embodiments, R4 is p-trifluoromethylphenyl. In some embodiments R4 is alkyl substituted.

In some embodiments, R3 is an aryl ring system, including phenyl, which can be optionally substituted. In some embodiments R3 is benzyl. In some embodiments R2 is alkyl.

In certain embodiments R2 is iso-propyl.

In some embodiments, R2 and R3 are taken together to form a N,N′-substituted piperazine.

In certain embodiments, R2 and R3 form a N,N′-{p-trifluorophenyl-substituted phenyl}piperazine.

In some embodiments, R1 is naphthalene and R4 is hydrogen. In certain embodiments, R1 is a substituted naphthalene and R4 is hydrogen. In some embodiments, R1 is naphthalene, R4 is hydrogen, and n is 2. In certain embodiments, R1 is a substituted naphthalene, R4 is hydrogen, and n is 2.

In some embodiments, R1 is aryl and R4 is hydrogen. In certain embodiments, R1 is a substituted aryl and R4 is hydrogen. In some embodiments, R1 is aryl, R4 is hydrogen, and n is 2. In certain embodiments, R1 is a substituted aryl, R4 is hydrogen, and n is 2.

In some embodiments, the compound is selected from 3-(benzyl(isopropyl)amino)-1-(naphthalen-2-yl)propan-1-ol; 3-(4-(4-fluorophenyl)piperazin-1-yl)-1-(naphthalen-2-yl)propan-1-ol; 3-((3,4-difluorobenzyl)(isopropyl)amino)-1-(naphthalen-2-yl)propan-1-ol; and 3-(benzyl(isopropyl)amino)-2-methyl-1-(naphthalen-2-yl)propan-1-ol.

“Alkyl” in general, refers to an aliphatic hydrocarbon group which may be straight, branched or cyclic having from 1 to about 10 carbon atoms in the chain, and all combinations and subcombinations of ranges therein. The term “alkyl” includes both “unsubstituted alkyls” and “substituted alkyls,” the latter of which refers to alkyl moieties having substituents replacing a hydrogen on one or more carbons of the backbone. In preferred embodiments, a straight chain or branched chain alkyl has 12 or fewer carbon atoms in its backbone (e.g., C₁-C₁₂ for straight chain, C₃-C₁₂ for branched chain), and more preferably 6 or fewer, and even more preferably 4 or fewer. Likewise, preferred cycloalkyls have from 3-10 carbon atoms in their ring structure, and more preferably have 5, 6 or 7 carbons in the ring structure. Unless the number of carbons is otherwise specified, “lower alkyl” as used herein means an alkyl group, as defined above, but having from one to ten carbons, more preferably from one to six carbon atoms in its backbone structure, and even more preferably from one to four carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl. Alkyl substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “aryl,” alone or in combination, means a carbocyclic aromatic system containing one, two or three rings wherein such rings may be attached together in a pendent manner or may be fused. The term “aryl” embraces aromatic radicals such as phenyl, naphthyl, tetrahydronapthyl, indane and biphenyl, and includes carbocyclic aryl, heterocyclic aryl and biaryl groups, all of which may be optionally substituted. The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “biaryl” represents aryl groups which have 5-14 atoms containing more than one aromatic ring including both fused ring systems and aryl groups substituted with other aryl groups. Such groups may be optionally substituted. Suitable biaryl groups include naphthyl and biphenyl. The term “carbocyclic” refers to cyclic compounds in which all of the ring members are carbon atoms. Such rings may be optionally substituted. The compound can be a single ring or a biaryl ring. The term “cycloalkyl” embraces radicals having three to ten carbon atoms, such as cyclopropyl, cyclobutyl, cyclopentyl, cyclohexyl, cycloheptyl and norboryl. Such groups may be substituted.

“Heterocyclic” aryl or “heteroaryl” groups are groups which have 5-14 ring atoms wherein 1 to 4 heteroatoms are ring atoms in the aromatic ring and the remainder of the ring atoms being carbon atoms. Suitable heteroatoms include oxygen, sulfur, and nitrogen. Suitable heteroaryl groups include furanyl, thienyl, pyridyl, pyrrolyl, N-lower alkyl pyrrolyl, pyridyl-N-oxide, pyrimidyl, pyrazinyl, imidazolyl, indolyl and the like, all optionally substituted. The term “heterocyclic” refers to cyclic compounds having as ring members atoms of at least two different elements. The compound can be a single ring or a biaryl. Heterocyclic groups include, for example, thiophene, benzothiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro chloro or bromo to provide “haloalkoxy” or “haloalkoxyalkyl” radicals. Examples of “alkoxy” radicals include methoxy, butoxy and trifluoromethoxy.

As used herein, the term “halogen” designates —F, —Cl, —Br or —I; the term “sulfhydryl” means —SH; and the term “hydroxyl” means —OH.

The term “methyl” refers to the monovalent radical —CH₃, and the term “methoxyl” refers to the monovalent radical —CH₂OH.

The term “aralkyl” or “arylalkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The terms “ortho”, “meta” and “para” apply to 1,2-, 1,3- and 1,4-disubstituted benzenes, respectively. For example, the names 1,2-dimethylbenzene and ortho-dimethylbenzene are synonymous.

As used herein, the definition of each expression, e.g., alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, the present invention relates to a compound represented by any of the structures outlined herein, wherein the compound is a single stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term stereochemically isomeric forms of compounds, as used herein, include all possible compounds made up of the same atoms bonded by the same sequence of bonds but having different three-dimensional structures which are not interchangeable, which the compounds may possess. Unless otherwise mentioned or indicated, the chemical designation of a compound encompasses the mixture of all possible stereochemically isomeric forms that the compound can take. The mixture can contain all diastereomers and/or enantiomers of the basic molecular structure of the compound. All stereochemically isomeric forms of the compounds both in pure form or in admixture with each other are intended to be embraced within the scope of the present invention.

Some of the compounds may also exist in their tautomeric forms. Such forms although not explicitly indicated in the above formula are intended to be included within the scope of the present invention.

It should be appreciated that in any of the aspects or embodiments described herein, the γ-secretase inhibitor compound(s) may be provided in any suitable stereoisomeric form, and/or any suitable polymorphic form, and/or pharmaceutically acceptable acid or base addition salt form, and in a therapeutically effective amount. Also, in any one of the aspects or embodiments described herein, the subject with neurodegenerative disorders and/or Alzheimer's disease may suffer from any γ-secretase-related or β-amyloid-associated disorder. The subject may be human. The effective amount of any one or more compounds may be from about 10 ng/kg of body weight to about 1000 mg/kg of body weight, and the frequency of administration may range from once a day to once a month. However, other dosage amounts and frequencies also may be used as the invention is not limited in this respect. It should be appreciated that one or more compounds and/or compositions of the invention may be used alone or in combination with one or more additional compounds or compositions to treat a subject that has Alzheimer's disease or that is at risk of developing Alzheimer's disease. In some embodiments, an additional compound may be an alternative inhibitor of gamma secretase (e.g., Flurbiprofen). In some embodiments, an additional compound may be a compound that is therapeutically useful for treating Alzheimer's disease or symptoms thereof (e.g., an acetyl-cholinesterase inhibitor, for example Aricept; an anti-depressive agent, for example rivastigmine; an NSAID; or any combination thereof). A combination therapy may involve combining one or more (e.g., 1, 2, 3, 4, 5, 6, 7, 8, 9, 10, or more) compounds of the invention with one or more (e.g., 1, 2, 3, 4, 5, 6, 7, 8, 9, 10, or more) additional compounds described herein. It should be appreciated that combination therapies may include compositions comprising one or more compounds and/or administering one or more compounds in combination (e.g., together, separately but according to a coordinated regimen, etc.).

It should be appreciated that compounds or compositions of the invention may be administered in an amount effective to treat a neurological disorder such as Alzheimer's in a subject. As used herein, the term treat refers to prophylactic and/or therapeutic applications. In some embodiments, a treatment may prevent the onset or development of disease or disease symptoms in a subject at risk of the disease (e.g., in a subject with a family history of Alzheimer's, a subject with early symptoms of Alzheimer's, a subject of an age associated with a higher risk for Alzheimer's, or a subject with any other risk factor for Alzheimer's, or a subject with any combination of two or more risk factors described herein). In some embodiments, a treatment may prevent or reduce the progression of the disease in a subject diagnosed as having Alzheimer's. In some embodiments, a treatment may promote disease regression. In preferred embodiments, the subject is a human.

Table 1 illustrates specific non-limiting embodiments of compounds of the invention. Non-limiting methods of synthesizing (e.g., method A or B) and of assaying the compounds listed in Table 1 are described in the Examples below. However, it should be appreciated that the methods described in the Examples are generally applicable to compounds of the invention and are not limited to those described in Table 1.

Table 2 illustrates specific non-limiting embodiments of compounds of the invention. Non-limiting methods of synthesizing (e.g., method A or B) and of assaying the compounds listed in Table 2 are described in the Examples below. However, it should be appreciated that the methods described in the Examples are generally applicable to compounds of the invention and are not limited to those described in Table 2.

It should be appreciated that the compounds described in tables 1 and 2 may be used alone or in combination according to methods of the invention. For example, one or more of these compounds may be provided in a pharmaceutical formulation (e.g., with a physiologically acceptable carrier) and administered to a subject to inhibit γ-secretase activity, and/or prevent, and/or treat one or more diseases or conditions described herein (e.g., AD).

TABLE 1
(I)

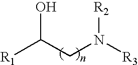

| Ex. | R₁ | n | R₂ | R₃ | Method |
|---|---|---|---|---|---|
| 1 | 2-naphthalene | 2 | —CH(CH₃)₂ | —CH₂Ph | A, |
| | | | | | B |
| 2 | 2-naphthalene | 2 | 1-(4-methoxy- | — | A |
| | | | pheny1)-4- | | |
| | | | piperazine | | |
| 3 | 2-naphthalene | 2 | 4-phenyl- | — | A |
| | | | piperazine | | |
| 4 | 2-naphthalene | 2 | —CH(CH₃)₂ | —CH₂Ph | A |
| 5 | 2-naphthalene | 2 | —CH₂Ph | —CH₂Ph | A |
| 6 | 2-naphthalene | 2 | azocane | — | A |
| 7 | 2-naphthalene | 2 | cyclohexane | cyclohexane | A |
| 8 | 2-naphthalene | 2 | —CH₃ | —CH₂Ph | A |
| 9 | 2-naphthalene | 2 | —CH₂CH(CH₃)₂ | —CH₂CH(CH₃)₂ | A |
| 10 | 2-naphthalene | 2 | —H | -Ph | A |
| 11 | 2-naphthalene | 2 | —C(CH₃)₃ | —CH₂Ph | A |
| 12 | 2-naphthalene | 2 | —CH₂CH₃ | —CH₂Ph | A |
| 13 | 2-naphthalene | 2 | -4-(3- | — | A |
| | | | (trifluoromethyl)- | | |
| | | | phenyl)- | | |
| | | | piperazine | | |
| 14 | 2-naphthalene | 2 | 1-(4-fluoro- | — | A |
| | | | phenyl)-4- | | |
| | | | piperazine | | |
| 15 | 2-naphthalene | 2 | —CH₃ | —CH₂CH₂Ph | A |
| 16 | 2-naphthalene | 2 | 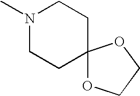 | — | A |
| 17 | 2-naphthalene | 2 | —CH(CH₃)₂ | —CH₂Ph(3,4-Cl) | A |
| 18 | 2-naphthalene | 2 | —CH(CH₃)₂ | —CH₂Ph(3,4-F) | A |
| 19 | 2-naphthalene | 2 | —CH(CH₃)₂ | —CH₂Ph | A |
| 20 | 2-naphthalene | 2 | —CH(CH₃)₂ | -Ph | A |
| 21 | 2-naphthalene | 2 | —H | -6-(2,3- | A |
| | | | | dihydrobenzo- | |
| | | | | [b]- | |
| | | | | [1,4]dioxine) | |
| 22 | 2-naphthalene | 2 | —H | -Ph-(4- | A |
| | | | | cyclohexane) | |
| 23 | 2-naphthalene | 2 | —H | -Ph(3,4-Cl) | A |
| 24 | 2-naphthalene | 2 | —H | -Ph(4-CF₃) | A |
| 25 | 2-naphthalene | 2 | —H | -Ph(4-OMe) | A |
| 26 | 2-naphthalene | 2 | —CH₃ | -Ph(4-OPh) | A |
| 27 | 2-naphthalene | 2 | —CH(CH₃)₂ | -Ph(4-Cl) | A |
| 28 | 2-naphthalene | 2 | —H | -Ph(2,5-F) | A |
| 29 | 2-naphthalene | 2 | —CH(CH₃)₂ | —CH₂Ph(4-OMe) | A |
| 30 | 4-tert-butyl- | 2 | —CH(CH₃)₂ | —CH₂Ph | A |
| | phenyl | | | | |
| 31 | 2-napthalene | 2 | —CH(CH₃)₂ | —CH₂Ph(3,4-F) | A |
| 32 | 2-napthalene | 2 | —H | -Ph(4-OEt) | A |
| 33 | 2-napthalene | 2 | —H | -Ph(3-Cl, 4-F) | A |
| 34 | 2-napthalene | 2 | —CH(CH₃)₂ | —CH₂Ph(4-CN) | B |
| 35 | 2-napthalene | 2 | —CH(CH₃)₂ | —CH₂Ph(3,5- | B |
| | | | | OMe) | |
| 36 | 2-napthalene | 2 | —CH(CH₃)₂ | —CH₂Ph(3-CN) | B |
| 37 | 2-napthalene | 2 | cyclopropane | —CH₂Ph | B |
| 38 | 4-fluoro- | 2 | —CH(CH₃)₂ | —CH₂Ph | B |
| | phenyl | | | | |
| 39 | -Ph | 2 | -4-fluorophenyl- | — | B |
| | (4-CF₃) | | piperazine | | |
| 40 | 4-tert- | 2 | -4-fluorophenyl- | — | B |
| | butyl- | | piperazine | | |
| | benzene | | | | |
| 41 | -Ph | 2 | —CH(CH₃)₂ | —CH₂Ph | B |
| | (4-CF₃) | | | | |
| 42 | 4-tert- | 2 | —CH(CH₃)₂ | —CH₂Ph(3,5-F) | B |
| | butyl- | | | | |
| | phenyl | | | | |
| 43 | 2-naphtha- | 2 | —CH(CH₃)₂ | —CH₂Ph(3-Cl, | B |
| | lene | | | 4-F) | |
| 44 | 2-naphtha- | 2 | —CH₃ | —CH₂Ph | B |
| | lene | | | | |
| 45 | 1-naphtha- | 2 | —CH(CH₃)₂ | —CH₂Ph | B |
| | lene | | | | |
| 46 | 2-naphtha- | 2 | —CH(CH₃)₂ | —CH₂Ph | B |
| | lene | | | | |
| 47 | 2-naphtha- | 2 | -4-phenyl- | — | B |
| | lene | | piperazine | | |
| 48 | -Ph | 2 | —CH(CH₃)₂ | —CH₂Ph | B |
| | (3-F, | | | | |
| | 4-CH₃) | | | | |
| 49 | -Ph(2,4,6- | 2 | —CH(CH₃)₂ | —CH₂Ph | B |
| | CH₃) | | | | |
| 50 | Ph-(4- | 2 | —CH(CH₃)₂ | —CH₂Ph | B |
| | tetrahydro- | | | | |
| | 2H- | | | | |
| | pyran | | | | |
| 51 | 2-naphtha- | 2 | —CH(CH₃)₂ | —CH₂Ph | B |
| | lene- | | | | |
| | (6-OMe) | | | | |
| 52 | -4-phenoxy | 2 | —CH(CH₃)₂ | —CH₂Ph | B |
| | phenyl | | | | |
| 53 | 3-(benzyl- | 2 | —CH(CH₃)₂ | —CH₂Ph | B |
| | oxy)- | | | | |
| | phenyl | | | | |
| 54 | Ph(3-F, | 2 | —CH(CH₃)₂ | —CH₂Ph | B |
| | 4-Me) | | | | |
| 55 | Ph(3-F, | 2 | —CH(CH₃)₂ | —CH₂Ph | B |
| | 5-Cl) | | | | |
| 56 | -Ph(2, | 2 | —CH(CH₃)₂ | —CH₂Ph | B |
| | 6-Me) | | | | |
| 57 | -Ph(3,5- | 2 | —CH(CH₃)₂ | —CH₂Ph | B |
| | CF₃) | | | | |
| 58 | -Ph(4- | 2 | —CH(CH₃)₂ | —CH₂Ph | B |
| | OCF₃) | | | | |
| 59 | -Ph(3,4- | 2 | —CH(CH₃)₂ | —CH₂Ph | B |
| | OMe) | | | | |
| 60 | -Ph(3-F, | 2 | —CH(CH₃)₂ | —CH₂Ph | B |
| | 4-OMe) | | | | |
| 61 | 2-(piperidin- | 2 | —CH(CH₃)₂ | —CH₂Ph | B |
| | 1-ylmethyl)- | | | | |
| | phenyl | | | | |
| 62 | 1-(benzo[d]- | 2 | —CH(CH₃)₂ | —CH₂Ph | B |
| | [1,3]-dioxol- | | | | |
| | 5-yl) | | | | |
| 63 | -Ph(3,4-Me) | 2 | —CH(CH₃)₂ | —CH₂Ph | B |
| 64 | -Ph(4-CN) | 2 | —CH(CH₃)₂ | —CH₂Ph | B |
| 65 | 2-naphtha- | 2 | —CH(CH₃)₂ | —CH₂Ph(3- | B |
| | lene | | | CO₂H) | |
| 66 | 2-naphtha- | 2 | —CH(CH₃)₂ | —CH₂Ph(4- | B |
| | lene | | | CO₂H) | |
| 67 | -Ph(4- | 2 | —CH(CH₃)₂ | —CH₂Ph | B |
| | CO₂H) | | | | |
| 68 | 6-quinoline- | 2 | -4-(4-fluoro- | — | B |
| | (2-Me) | | phenyl)- | | |
| | | | piperazine | | |
| 69 | 2-benzo- | 2 | —H | -Ph(4-OPh) | A |
| | furan- | | | | |
| | (3-Me) | | | | |
| 70 | 2-benzo- | 2 | —CH(CH₃)₂ | —CH₂Ph | A |
| | furan- | | | | |
| | (7-OMe) | | | | |
| 71 | 2-naphtha- | 2 | —CH(CH₃)₂ | —CH₂-2- | B |
| | lene | | | pyridine- | |
| | | | | (6-Me) | |
| 72 | 2-naphtha- | 2 | —CH(CH₃)₂ | -Ph(4-OPh) | B |
| | lene | | | | |
| 73 | 2-naphtha- | 2 | —CH(CH₃)₂ | —COPh | B |
| | lene | | | | |
| 74 | 2-naphtha- | 2 | —CH(CH₃)₂ | CH₂Ph(2-CO₂H) | B |
| | lene | | | | |
| 75* | 2-naphtha- | 2 | —CH(CH₃)₂ | —CH₂Ph | B |
| | lene | | | | |
| 76* | 2-naphtha- | 2 | —CH(CH₃)₂ | —CH₂Ph | B |
| | lene | | | | |
| 77 | 2-naphtha- | 2 | —H | -Ph(4-OPh) | A |
| | lene | | | | |
| 78 | 2-naphtha- | 2 | —H | -Ph(3-F, 4-Me) | A |
| | lene | | | | |
| 79 | -Ph(4-F) | 2 | —H | -Ph(4-OEt) | A |
| 80 | -Ph(4-Cl) | 2 | —H | -Ph(4-OMe) | A |
| 81 | 2-naphtha- | 2 | -4-phenyl- | — | A |
| | lene | | piperazine | | |
| 82 | 2-naphtha- | 2 | —CH(CH₃)₂ | —CH₂Ph | |
| | lene | | | | |
| 83 | 2-naphtha- | 2 | —H | -Ph(4-Cl, | A |
| | lene | | | 3-NO₂) | |
| 84* | 2-naphtha- | 2 | —CH(CH₃)₂ | —CH₂Ph | A, B |
| | lene | | | | |
| 85* | 2-naphtha- | 2 | —CH(CH₃)₂ | —CH₂Ph | A, B |
| | lene | | | | |
| 86* | 2-naphtha- | 2 | —CH(CH₃)₂ | —CH₂Ph | A |
| | lene | | | | |
| | 6-[(N-benzyl- | | | | |
| | N-isopropyl-1- | | | | |
| | amino-3- | | | | |
| | hydroxy-)-3- | | | | |
| | propane] | | | | |
| 87* | 2-naphtha- | 2 | —CH(CH₃)₂ | —CH₂Ph | A |
| | lene | | | | |
| 88* | 2-naphtha- | 2 | —CH(CH₃)₂ | —CH₂Ph | A |
| | lene | | | | |
| 89 | 2-naphtha- | 2 | -4-(4-fluoro- | — | A |
| | lene | | phenyl)- | | |
| | | | piperazine | | |
| 90 | 2-naphtha- | 2 | -4-(4-fluoro- | — | A |
| | lene | | phenyl)- | | |
| | | | piperazine | | |
| 91 | 4-benzoic | 2 | —CH(CH₃)₂ | -6-methyl- | B |
| | acid | | | pyridin-2- | |
| | | | | yl)-methyl) | |
| 92 | 4- | 2 | —CH(CH₃)₂ | —CH₂-2- | B |
| | carboxy- | | | picolinic | |
| | phenyl | | | acid | |
| 93 | 4-benzoic | 2 | —CH(CH₃)₂ | —CH₂Ph(3,4-F) | B |
| | acid | | | | |
| 94 | 3- | 2 | —CH(CH₃)₂ | -Ph(4-OPh) | B |
| | methyl- | | | | |
| | benzo-2- | | | | |
| | furan-2-yl | | | | |
| 95 | 5-picolinic | 2 | —CH(CH₃)₂ | —CH₂Ph(3-F, | B |
| | acid | | | 5-OMe) | |
| 96 | (6-trifluoro- | 2 | —CH(CH₃)₂ | —CH₂Ph(3,4-F) | B |
| | methyl)- | | | | |
| | pyridin- | | | | |
| | 3-yl | | | | |
| 97 | 3-(5-meth- | 2 | —CH(CH₃)₂ | —CH₂Ph(3,4-F) | B |
| | oxy-2- | | | | |
| | benzoic- | | | | |
| | acid) | | | | |
| 98 | 2-naphtha- | 2 | —CHCH₂(CH₃)₂ | —CH₂Ph(3,4-F) | B |
| | lene | | | | |
| 99 | 1-(4- | 2 | —CHCH₂(CH₃)₂ | —CH₂Ph(3,4-F) | B |
| | trifluoro- | | | | |
| | methyl)- | | | | |
| | phenyl | | | | |
| 100 | 1-(4-tert- | 2 | —CHCH₂(CH₃)₂ | —CH₂Ph(3,4-F) | B |
| | butyl)- | | | | |
| | phenyl | | | | |
| 101 | 1-(4-tert- | 2 | —CHCH₂(CH₃)₂ | —CH₂Ph(3,4-F) | B |
| | butyl)- | | | | |
| | phenyl | | | | |
| 102 | 1-(4- | 2 | —CH(CH₃)₂ | —CH₂Ph(3,4-F) | B |
| | trifluoro- | | | | |
| | methyl)- | | | | |
| | phenyl | | | | |
| 103 | 2-naphtha- | 2 | —CHCH₂(CH₃)₂ | —CH₂Ph(3,4-Cl) | B |
| | lene | | | | |
| 104 | 4-methyl- | 2 | —CHCH₂(CH₃)₂ | —CH₂Ph | B |
| | naphthalen- | | | | |
| | 1-yl | | | | |
| 105 | 4-methyl- | 2 | —CH(CH₃)₂ | —CH₂Ph | B |
| | naphthalen- | | | | |
| | 1-yl | | | | |
| 106 | 1-(4- | 2 | —CHCH₂(CH₃)₂ | —CH₂Ph(3,4-F) | B |
| | trifluoro- | | | | |
| | methyl)- | | | | |
| | phenyl | | | | |
| 107 | 1-(4- | 2 | —CHCH₂CH₂(CH₃)₂ | —CH₂Ph(3,4-F) | B |
| | trifluoro- | | | | |
| | methyl)- | | | | |
| | phenyl | | | | |
| 108 | 2-naphtha- | 2 | —CHCH₂CH₂(CH₃)₂ | —CH₂Ph(3,4-F) | B |
| | lene | | | | |
| 109 | 1-(4-tert- | 2 | —CHCH₂CH₂(CH₃)₂ | —CH₂Ph(3,5-F) | B |
| | butyl)- | | | | |
| | phenyl | | | | |
| 110 | 2-naphtha- | 2 | —CHCH₂CH₂(CH₃)₂ | —CH₂Ph(3,4-Cl) | B |
| | lene | | | | |
| 111 | 4-methyl- | 2 | —CHCH₂CH₂(CH₃)₂ | —CH₂Ph | B |
| | naphthalen- | 2 | | | |
| | 1-yl | | | | |
| 112 | 6-quinoline- | 2 | —CH(CH₃)₂ | —CH₂Ph(3,4-F) | B |
| | 2-carboxy- | | | | |
| | lic acid | | | | |
| 113 | 2- | 2 | —CH(CH₃)₂ | —CH₂Ph(3,4-F) | B |
| | methyl- | | | | |
| | quinolin- | | | | |
| | 6-yl | | | | |
| 114 | 4-benzo- | 2 | —CH(CH₃)₂ | —CH₂Ph(3,4-F) | B |
| | nitrile | | | | |
| 115 | 4-(2-fluoro- | 2 | —CH(CH₃)₂ | —CH₂Ph(3,4-F) | B |
| | benzonitrile) | | | | |
| 116 | 5-chloro- | 2 | —CH(CH₃)₂ | —CH₂Ph(3,4-F) | B |
| | thiophen- | | | | |
| | 2-yl | | | | |

75* (R)-enantiomer; 76* (S)-enantiomer; 84* Hydroxyl is methylated to the corresponding ether -Ome using butyl lithium and methyl ioidide under standard alkylating conditions; 85* Hydroxyl is arylated to the corresponding ether -OPh(4-CF₃) using standard alkyating conditions; 86* The compound is dimeric with a C2 axis of symmetry; 87* The compound contains a syn-methyl substituent a to the hydroxyl substituent; 88* The compound contains an anti-methyl substituent a to the hydroxyl substituent; 89* (1S,2S)-enantiomer. The compound contains an anti-methyl substituent a to the hydroxyl substituent; 90* (1S,2R)-enantiomer. The compound contains a syn-methyl substituent a to the hydroxyl substituent;

In some embodiments, a compound may be of the general formula described herein (e.g., in (1), (I), or (2)), but excluding any one or more (e.g., 1, 2, 3, 4, 5, 6, 7, 8, 9, 10, or more) of the specific compounds listed in Table 1 or 2 herein (e.g., excluding Ex. 1 in Table 1).

Accordingly, in some embodiments, a composition of the invention includes a compound of formula (1), (I), or (2) wherein the compound is not one or more of the specific compounds described in Tables 1 and 2. In some embodiments, the compound is a compound of formula (1), (I), or (2) but is not one or more of compounds 1-116 of Table 1 or Table 2. For example, a composition may include a compound of formula (1), (I), or (2) wherein the compound is not Example 1 of Table 1 or Table 2.

In some embodiments a composition comprises a compound of formula (1), (I), or (2), wherein the compound does not have an isopropyl, and/or has a substituted benzyl, and/or has a naphthalene substituent.

In some aspects of the invention, compositions comprise compounds which comprise naphthalene substituents. In other aspects of the invention, compositions comprise compounds with alkyl substituents, wherein the alkyl substituent is methyl, ethyl, n-propyl, isopropyl, tert-butyl, cyclopropyl, cyclohexyl, 2-methylpropyl, or 2-methylbutyl. In other aspects of the invention, compositions comprise compounds with aryl substituents, wherein the aryl substituent is naphthyl, substituted naphthyl, phenyl, substituted phenyl, pyridyl, substituted pyridyl, quinoline, substituted quinoline, thiophene, substituted thiophene, benzyl, substituted benzyl, phenolic, substituted phenolic, O-alkyl phenolic, O-aryl phenolic, furan, substituted furan, benzofuran, or substituted benzofuran. It should be appreciated that substituted groups described herein may be substituted with one or more halogens, alkyl, aryl, heteroatom containing groups, or other substituents.

Another aspect of the invention includes molecules that are dimeric forms of the compounds described herein. In some embodiments, one or more compounds may be a dimer with a C2 axis of symmetry centered at R1. Such examples are exemplified by compound number 86 with its C2 axis of symmetry running through its naphthalene (R1) substituent. However, any compound described herein may be dimerized in a similar fashion. In some embodiments, it is expected that dimeric forms may be more potent and be useful at lower concentrations than their monomeric counterparts.

In some embodiments, a compound of the invention inhibits γ-secretase activity by at least 10% (e.g., by at about 50%, by about 75%, by about 80%, by about 90%, by about 95%, or more, for example completely inhibits) at a concentration of about 100 μM when assayed in an assay described herein (e.g., the C-100 assay). Accordingly, in some embodiments a compound of the invention does not have less than 10% inhibitory activity when assayed at a concentration of about 100 μM in an assay described herein (e.g., the C-100 assay). In some embodiments, the inhibitory activity of a compound is selective for gamma-secretase mediated cleavage of APP relative to Notch protein. Accordingly, in some embodiments, a compound of the invention inhibits gamma-secretase activity against APP (e.g., by at least 10%, by at about 50%, by about 75%, by about 80%, by about 90%, by about 95%, or more, for example completely inhibits) to a greater extent than it inhibits gamma-secretase activity against the Notch protein. In some embodiments, a compound of the invention that inhibits APP cleavage does not inhibit Notch cleavage significantly (e.g., no inhibition of Notch cleavage, or enhanced Notch cleavage, is observed using an assay described herein, for example the N-100 assay or other assay). In some embodiments, an inhibitor is at least 5 fold (e.g., at least 10 fold, at least 100 fold, etc.) more selective for inhibiting APP cleavage relative to Notch cleavage. In some embodiments, a compound of the invention has an IC50 of between 1 and 10 μM (e.g., about 1 μM, about 2 μM, about 3 μM, about 4 μM, about 5 μM, about 6 μM, about 7 μM, about 8 μM, about 9 μM, or about 10 μM) for APP but a higher IC50 (e.g., about 10-100 fold or higher) IC50 for Notch. Accordingly, in some embodiments, the IC50 for Notch may be about 25 μM, about 50 μM, about 75 μM, about 100 μM or higher. However, it should be appreciated that a compound of the invention may be selective even if it has a higher IC50 for APP (e.g., higher than 10 μM, higher than 50 μM, etc.), provided that the IC50 for Notch is relatively higher (e.g., higher than 100 μM, higher than 500 μM, etc.).

Another aspect of the invention provides an article of manufacture (e.g., a kit) comprising packaging material and a γ-secretase inhibitor compound, wherein the article of manufacture further comprises a label or package insert indicating that the γ-secretase inhibitor compound can be administered to a subject for treating neurodegenerative disorders. In a preferred embodiment the subject is a human. In some embodiments, an article of manufacture may include two or more compounds or compositions of the invention alone or along with one or more additional compounds or compositions that are useful for treating Alzheimer's disease as described herein.

In methods of the invention, the term “subject with neurodegenerative disorders” refers to a subject that is affected by or at risk of developing neurodegenerative disorders (e.g. predisposed, for example genetically predisposed, to developing Alzheimer's disease) and/or any neurodegenerative disorders characterized by pathological aggregations of β-amyloid plaques or peptide fragments.

The phrase “therapeutically-effective amount” as used herein means that amount of a compound, material, or composition comprising a compound of the present invention which is effective for producing some desired therapeutic effect in a subject at a reasonable benefit/risk ratio applicable to any medical treatment. Accordingly, a therapeutically effective amount prevents, minimizes, or reverses disease progression associated with neurodegenerative disorders. Disease progression can be monitored by clinical observations, laboratory and neuroimaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The “pharmaceutically acceptable acid or base addition salts” mentioned herein are meant to comprise the therapeutically active non-toxic acid and non-toxic base addition salt forms that the compounds are able to form. The compounds that have basic properties can be converted into their pharmaceutically acceptable acid addition salts by treating the base form with an appropriate acid. Appropriate acids include, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The compounds that have acidic properties can be converted into their pharmaceutically acceptable base addition salts by treating the acid form with a suitable organic or inorganic base. Appropriate base salt forms include, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds are able to form. Examples of such forms are e.g. hydrates, alcoholates and the like.

The methods and structures described herein relating to compounds and compositions of the invention also apply to the pharmaceutically acceptable acid or base addition salts and all stereoisomeric forms of these compounds and compositions.

Some of the compounds and hydrates of these compounds may exist in one or several polymorphic forms, resulting from reversible and irreversible alterations in their associated crystalline structures. Certain compounds may have dissolution rates that vary according to their polymorphic crystalline structure.

Contemplated equivalents of the compounds described above include compounds which otherwise correspond thereto, and which have the same general properties thereof (e.g., functioning as anti-γ-secretase inhibitor compounds), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants, which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As set out herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routs of administration include sublingual, intramuscular, and transdermal administrations.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In some embodiments, a compound or pharmaceutical composition of the invention is chronically provided to a subject with neurodegenerative disorders. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the subject with neurodegenerative disorders. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition) as described above.

The compounds according to the invention may be formulated for administration in any convenient way for use in human or veterinary medicine, by analogy with other pharmaceuticals.

According to the invention, compounds for treating neurological conditions or diseases can be formulated or administered using methods that help the compounds cross the blood-brain barrier (BBB). The vertebrate brain (and CNS) has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood.

The unique morphologic characteristics of the brain capillaries that make up the BBB are: (a) epithelial-like high resistance tight junctions which literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

In one aspect of the invention, γ-secretase inhibitor compounds that cross the BBB are particularly useful for treating subjects with neurodegenerative disorders. In one embodiment, it is expected that γ-secretase inhibitors that are non-charged (e.g., not positively charged) and/or non-lipophilic may cross the BBB with higher efficiency than charged (e.g., positively charged) and/or lipophilic compounds. Therefore it will be appreciated by a person of ordinary skill in the art that some of the compounds of the invention might readily cross the BBB. Alternatively, the compounds of the invention can be modified, for example, by the addition of various substitutuents that would make them less hydrophilic and allow them to more readily cross the BBB.

Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference).

Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl and primary amine groups on the drug to make it more lipid-soluble and therefore more easily able to cross the blood-brain barrier.

Another approach to increasing the permeability of the BBB to drugs involves the intra-arterial infusion of hypertonic substances which transiently open the blood-brain barrier to allow passage of hydrophilic drugs. However, hypertonic substances are potentially toxic and may damage the blood-brain barrier.

Peptide compositions of the invention may be administered using chimeric peptides wherein the hydrophilic peptide drug is conjugated to a transportable peptide, capable of crossing the blood-brain barrier by transcytosis at a much higher rate than the hydrophilic peptides alone. Suitable transportable peptides include, but are not limited to, histone, insulin, transferrin, insulin-like growth factor I (IGF-I), insulin-like growth factor II (IGF-II), basic albumin and prolactin.

Antibodies are another method for delivery of compositions of the invention. For example, an antibody that is reactive with a transferrin receptor present on a brain capillary endothelial cell, can be conjugated to a neuropharmaceutical agent to produce an antibody-neuropharmaceutical agent conjugate (U.S. Pat. No. 5,004,697 incorporated herein in its entirety by reference). The method is conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point of between about 8.0 to 11.0 (U.S. Pat. No. 5,527,527 incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion.

The permeability of the blood brain barrier can be increased by administering a blood brain barrier agonist, for example bradykinin (U.S. Pat. No. 5,112,596 incorporated herein in its entirety by reference), or polypeptides called receptor mediated permeabilizers (RMP) (U.S. Pat. No. 5,268,164 incorporated herein in its entirety by reference). Exogenous molecules can be administered to the host's bloodstream parenterally by subcutaneous, intravenous or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g. between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g. bradykinin) can be administered before or at the same time as an intravenous injection of a drug. Thus, the term “co administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system.

In other embodiments, compounds of the invention can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and the γ-secretase inhibitor (and optionally another drug). The carrier preferably is a normal component of the central nervous system and is inactive and harmless. The compound and/or drug, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. Pat. Nos. 4,939,174; 4,933,324; 5,994,932; 6,107,499; 6,258,836 and 6,407,137, the disclosures of which are incorporated herein by reference in their entirety.

The administration of the agents of the present invention may be for either prophylactic or therapeutic purpose. When provided prophylactically, the agent is provided in advance of disease symptoms such as any Alzheimer's disease symptoms. The prophylactic administration of the agent serves to prevent or reduce the rate of onset of symptoms. When provided therapeutically, the agent is provided at (or shortly after) the onset of the appearance of symptoms of actual disease. In some embodiments, the therapeutic administration of the agent serves to reduce the severity and duration of Alzheimer's disease.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood, unless otherwise indicated, to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

All references cited herein, including patents and published applications, are incorporated herein by reference. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

### EXAMPLES

### Example 1

Experimental Procedures Describing Two Methods for the Synthesis of Compound (I) The synthetic procedures described for the preparation of the compounds featured herein were generally applicable to all of the compounds described throughout this disclosure.

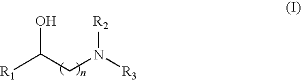

Method A—Description

Several Examples in Table 1 were synthesized using the synthetic route that is illustrated in Scheme 1, Method A. The addition of the appropriate and readily available vinyl Grignard reagents to aryl aldehyde (II) gave the desired vinyl alcohol (III). The Grignard reaction was quenched with 1N HCl and the resulting mixture was extracted with ethyl acetate. The resulting crude residue of vinyl alcohol was dissolved in methylene chloride and subjected to oxidation conditions using chromium (III) oxide and tert-butylhydroperoxide (TBHP) to give the α,β-unsaturated ketone (IV). Under neat reaction conditions, the Michael addition of the desired substituted amine to (IV) in the presence of a stoichiometric amount of solid LiClO₄ to ketone (IV) gave the corresponding Michael adduct (V) in high yield in a short reaction time. However, Michael adduct (V) could not be subjected to purification via silica gel because decomposition to ketone (IV) would result. (See Brown, G. R., Bamford, A. M., Bowyer, J., James, D. S., Rankine, N., Tang, E., Ton, V., Culbert, E. J. Bioorganic & Medicinal Chemistry Letters, 2000, 10, 575-579.) The crude adduct (V) was then subjected to reduction using sodium borohydride to provide the desired β-amino alcohol (I) in good yield.

Based on this sequence of reactions depicted in Scheme 1, several analogs of formula (I) have been synthesized via this synthetic route (e.g., Examples 1-33, 69-70, 77-81, and 83-90). It is worthy to note that liquid or solid amines, including primary and secondary amines, can be subjected to Michael addition to provide the desired products in good yields. Reaction times can vary depending on the type of amine used. For example, when (IV) was reacted with aniline, the reaction time was 16 hours and the yield was lower than when (IV) was reacted with N-isopropylbenzylamine in which case the reaction time was one hour and the yield was moderate.

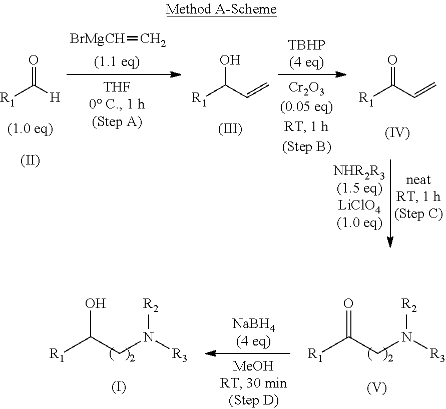

Method A—Procedures

### Preparation of 3-(benzyl(isopropyl)amino)-1-(naphthalen-2-yl)propan-1-ol

### Step A: Preparation of 1-(naphthalen-2-yl)prop-2-en-1-ol

In a 250 mL round-bottom flask, 22 mL of vinylmagnesium bromide (1.0 M in THF, 22 mmol, 1.11 eq) was added dropwise over 5 minutes to a solution of 2-naphthaldehyde (3.15 g, 19.8 mmol, 1.0 eq) in 100 mL of anhydrous THF at 0° C. After 1 hour, the reaction mixture was poured into a beaker that contained 100 mL of cold 1N HCl solution. The mixture was extracted with ethyl acetate (3×100 mL) and concentrated in vacuo to give crude 1-(naphthalen-2-yl)prop-2-en-1-ol which was used without further purification.

### Step B: Preparation of 1-(naphthalen-2-yl)prop-2-en-1-one

The 1-(naphthalen-2-yl)prop-2-en-1-ol that was obtained from Step A was dissolved in 100 mL CH₂Cl₂. Chromium(III) oxide (100 mg, 1.0 mmol, 0.05 eq) and 11.7 mL (˜80 mmol, 4.0 equiv) of TBHP (tert-butyl hydroperoxide solution, * 70% aqueous solution, Aldrich) was added to the alcohol solution in one portion. The color of the solution turned purple within about one minute after the addition of TBHP. The purple color disappeared in 15 minutes. After 1 hour of stirring at room temperature, 200 mL of H₂O was added and the reaction mixture was extracted with dichloromethane (CH₂Cl₂) (3×100 mL). Emulsion occurred but was cleared after the organic layer was washed with 100 mL of brine. The combined organic phases were then passed through a pad of silica gel to remove any insoluble material and then concentrated in vacuo to give 1-(naphthalen-2-yl)prop-2-en-1-one, a red residue.

### Step C: Preparation of 3-(benzyl(isopropyl)amino)-1-(naphthalen-2-yl)propan-1-one

Lithium perchlorate (2.13 g, 20.0 mmol, 1.0 eq) and N-isopropylbenzylamine (4.94 mL (30 mmol, 1.5 eq) were added to the red residue of 1-(naphthalen-2-yl)prop-2-en-1-one from Step B. A room temperature water bath was necessary to control the temperature of this very exothermic reaction. After the reaction mixture was stirred at room temperature for 1 hour, 100 mL of CH₂Cl₂ was added. Lithium perchlorate was removed by filtration and the organic layer was concentrated in vacuo to give crude 3-(benzyl(isopropyl)amino)-1-(naphthalen-2-yl)propan-1-one.

### Step D: Preparation of 3-(benzyl(isopropyl)amino)-1-(naphthalen-2-yl)propan-1-ol

The 3-(benzyl(isopropyl)amino)-1-(naphthalen-2-yl)propan-1-one was dissolved in 50 mL of methanol and sodium borohydride (3.02 g, 80 mol, 4.0 equiv) was added portionwise over a fifteen minute period. After 1 hour, the methanol was removed and the crude product was subjected to column chromatography on silica gel eluting with hexane:ethyl acetate (10:1) to yield 2.30 g of 3-(benzyl(isopropyl)amino)-1-(naphthalen-2-yl)propan-1-ol, a light red oil (35%).

¹H NMR (500 MHz, CDCl₃) δ 7.80 (m, 4H), δ 7.40 (m, 8H), δ 4.95 (dd, J₁=7.5 Hz, J₂=2.5 Hz, 1H), δ 3.84 (d, J=11 Hz, 1H), δ 3.42 (d, J=11 Hz, 1H), δ 3.14 (m, 1H), δ 2.90 (m, 1H), δ 2.70 (m, 1H), δ 1.94 (m, 1H), δ 1.83 (m, 1H), δ 1.17 (d, J=5.5 Hz, 3H), δ 1.02 (d, J=5.5 Hz, 3H).

Method B—Description

A second synthetic route was employed for the synthesis of compounds of formula (I). This route involved a one-pot-two-step procedure utilizing a 1,8-diazabicyclo[5.4.0]undec-7-ene-catalyzed Michael addition of amines (VII) to acrolein (VI) to give the desired β-amino aldehydes (VIII) in situ. (See Markó, I. E.; Chesney, A. Synlett, 1992, 275-278.) Treatment of (VIII) with readily available substituted aromatic Grignard reagents gave the desired β-amino alcohol (I) in good yield. Several analogs in Table 1 (e.g., Examples 34-68, 71-76, and 84-85) were synthesized by this one pot reaction procedure. This method does not work well when using primary amines (VII), but requires a shorter reaction time and limited purifications relative to Method A. Hence, method B is a very efficient synthetic route for the synthesis of compounds of formula (I).

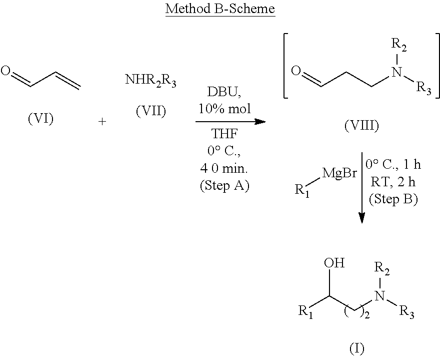

Method B—Procedures

### Preparation of 3-(benzyl(isopropyl)amino)-1-(naphthalen-2-yl)propan-1-ol

### Step A: Preparation of 3-(benzyl(isopropyl)amino)propanal

To a 50 mL round-bottom flask that contains N-benzylisopropylamine (2.4 mmol, 1.2 eq), tetrahydrofuran (15 mL) and acrolein (2.0 mmol, 1.0 eq), 1,8-diazabicyclo[5.4.0]undec-7-ene was added (0.2 mmol, 0.1 eq) at 0° C. and stirred for forty minutes to give 3-(benzyl(isopropyl)amino)propanal in situ.

### Step B: Preparation of 3-(benzyl(isopropyl)amino)-1-(naphthalen-2-yl)propan-1-ol

To the 3-(benzyl(isopropyl)amino)propanal solution from Step A, naphthalen-2-ylmagnesium bromide (2.4 mmol, 1.2 eq) was added dropwise at 0° C. After stirring at 0° C. for 1 hour and then stirred for two hours at room temperature, the reaction mixture was quenched with 20 mL of water. This mixture was then extracted with ethyl acetate (3×20 mL) and the combined organic phase was dried (MgSO₄), filtered, and concentrated in vacuo to give crude product. Purification by column chromatography (silica gel, hexane/ethyl acetate=10:1) gave the desired product, 3-(benzyl(isopropyl)amino)-1-(naphthalen-2-yl)propan-1-ol as an oil. (454 mg, 68%).

¹H NMR (500 MHz, CDCl₃) δ 7.80 (m, 4H), δ 7.40 (m, 8H), δ 4.95 (dd, J₁=7.5 Hz, J₂=2.5 Hz, 1H), δ 3.84 (d, J=11 Hz, 1H), δ 3.42 (d, J=11 Hz, 1H), δ 3.14 (m, 1H), δ 2.90 (m, 1H), δ 2.70 (m, 1H), δ 1.94 (m, 1H), δ 1.83 (m, 1H), δ 1.17 (d, J=5.5 Hz, 3H), δ 1.02 (d, J=5.5 Hz, 3H).

### Example 2

Cell Lines and Cultures—HeLa S3 cells, the Chinese hamster ovary (CHO) γ_{γ}-30 cell line (co-expressing human PS1, FLAG-Pen-2, and Aph1α2-HA), and the S-1 CHO cell line (co-expressing human PS1, FLAG-Pen-2, Aph1α2-HA, and NCT-GST) are cultured as described previously (Fraering, P. C., Ye, W., Strub, J. M., Dolios, G., LaVoie, M. J., Ostaszewski, B. L., Van Dorsselaer, A., Wang, R., Selkoe, D. J., and Wolfe, M. S. (2004) Biochemistry 43, 9774-9789, Kimberly, W. T., Esler, W. P., Ye, W., Ostaszewski, B. L., Gao, J., Diehl, T., Selkoe, D. J., and Wolfe, M. S. (2003) Biochemistry 42, 137-144, Fraering, P. C., LaVoie, M. J., Ye, W., Ostaszewski, B. L., Kimberly, W. T., Selkoe, D. J., and Wolfe, M. S. (2004) Biochemistry 43, 323-333).

### Example 3

Purification of γ-Secretase and in Vitro γ-Secretase Assays—The following procedures can be used to isolate γ-secretase and measure its enzymatic activity. The multistep procedure for the high grade purification of human γ-secretase from the S-1 cells is performed as described previously (Fraering, P. C., et al. (2004) Biochemistry 43, 9774-9789). In vitro γ-secretase assays using the recombinant APP-based substrate C100FLAG and the recombinant Notch-based substrate N100FLAG are performed as reported previously (Esler, W. P., Kimberly, W. T., Ostaszewski, B. L., Ye, W., Diehl, T. S., Selkoe, D. J., and Wolfe, M. S. (2002) Proc. Natl. Acad. Sci. U.S.A. 99, 2720-2725, Kimberly, W. T., et al. (2003) Biochemistry 42, 137-144). Basically, the proteolytic reaction mixtures contain C100FLAG and N100FLAG substrate at a concentration of 1 μm, purified γ-secretase solubilized in 0.2% CHAPSO/HEPES, pH 7.5, at 10-fold dilution from stock (stock=the M2 anti-FLAG-eluted fraction in the purification protocol from S-1 cells (Fraering, P. C., et al. (2004) Biochemistry 43, 9774-9789)), 0.025% phosphatidylethanolamine (PE), and 0.10% phosphatidylcholine (PC). All the reactions are stopped by adding 0.5% SDS, and the samples are assayed for Aβ40 and Aβ42 by ELISA as described (Xia, W., Zhang, J., Ostaszewski, B. L., Kimberly, W. T., Seubert, P., Koo, E. H., Shen, J., and Selkoe, D. J. (1998) Biochemistry 37, 16465-16471). The capture antibodies are 2G3 (to Aβ residues 33-40) for the Aβ40 species and 21F12 (to Aβ residues 33-42) for the Aβ42 species.

### Example 4

Western Blotting and Antibodies—The following assay can be used to determine the extent to which the compounds of interest modulate the cleavage of APP and the Notch receptor. For Western analysis of PS1-NTF, PS1-CTF, Aph1α2-HA, FLAG-Pen-2, and NCT-GST, the samples are run on 4-20% Tris-glycine polyacrylamide gels, transferred to polyvinylidene difluoride, and can be probed with Ab14 (for PS1-NTF, 1:2000; a gift of S. Gandy), 13A11 (for PS1-CTF, 5 μg/ml; a gift of Elan Pharmaceuticals), 3F10 (for Aph1α2-HA, 50 ng/ml; Roche Applied Science), anti-FLAG M2 (for FLAG-Pen-2, 1:1000; Sigma), or αGST antibodies (for NCT-GST, 1:3000; Sigma). Samples from the γ-secretase activity assays (above) are run on 4-20% Tris-glycine gels and can be transferred to polyvinylidene difluoride membranes to detect AICD-FLAG with anti-FLAG M2 antibodies (1:1000, Sigma) and NICD-FLAG with Notch Ab1744 antibody (1:1000, Cell Signaling Technology), which is selective for the N terminus of NICD; the same samples are transferred to nitrocellulose membranes to detect Aβ with the anti-Aβ 6E10 antibody. Levels of AICD-FLAG and NICD-FLAG are estimated by densitometry using AlphaEase/Spot Denso (Alpha Innotech Corp.).

### Example 5

Purified γ-Secretase and Binding to ATP-immobilized Resins—The following assay can be used to determine the extent to which the compounds of interest bind to ATP. The purified γ-secretase is diluted 10-fold from stock (Fraering, P. C., et al. (2004) Biochemistry 43, 9774-9789) in 50 mM HEPES buffer, pH 7.0, containing 0.2 or 1% CHAPSO, 150 mM NaCl, 5 mM MgCl₂, 5 mM CaCl₂ and can be incubated overnight, in the presence or absence of 50 mM ATP (Sigma), with ATP-agarose (ATP attached to agarose through the ribose hydroxyls, Sigma catalog number A-4793) or ATP-acrylamide (ATP attached to acrylamide through the γ-phosphate; Novagen catalog number 71438-3). Each resin is washed three times with 0.2 or 1% CHAPSO/HEPES buffer, and the bound proteins are collected in 2× Laemmli sample buffer, and can be resolved on 4-20% Tris-glycine gels, then transferred to polyvinylidene difluoride membranes to detect NCT-GST, PS1-NTF, Aph1-HA, PS1-CTF, and FLAG-Pent as described above.

### Example 6

Photoaffinity Labeling Experiments—The following assay can be used to determine the extent to which the compounds of interest inhibit the cleavage of APP. 8-Azido[γ-³²P]ATP (18 Ci/mmol) is purchased from Affinity Labeling Technology (Lexington, Ky.). For the photoaffinity labeling of the purified γ-secretase, the enzyme is diluted 10-fold from stock (Fraering, P. C., et al. (2004) Biochemistry 43, 9774-9789) in 50 mM HEPES buffer, pH 7.0, containing 0.2% CHAPSO, 150 mM NaCl, 5 mM MgCl₂, 5 mM CaCl₂, 0.025% PE, and 0.10% PC. The samples are exposed to UV light for 5 mM (hand-held UV lamp at 254 nm; UVP model UVGL-25) on ice, and the reaction is quenched with 1 mM dithiothreitol. The proteins are diluted in 0.5% CHAPSO/HEPES buffer and incubated overnight for affinity precipitation with GSH resin as described previously (Fraering, P. C., et al. (2004) Biochemistry 43, 9774-9789, Fraering, P. C., et al. (2004) Biochemistry 43, 323-333). The unbound nucleotides are removed by washing the resin three times and then the washed proteins are resuspended in Laemmli sample buffer. For the photoaffinity labeling of the purified γ-secretase followed by the BN-PAGE analysis, the enzyme is diluted in 0.1% digitonin/TBS, exposed to UV light for 5 min, and directly loaded onto a 5-13.5% BN-polyacrylamide gel. For the photoaffinity labeling of endogenous γ-secretase, HeLa S3 membranes (the equivalent of 3.0×10⁸ cells) are incubated with 22.5 μM 8-azido-[γ-³²P]ATP (10 μCi per reaction), 50 mM HEPES, pH 7.0, 150 mM NaCl, 5 mM MgCl₂, and 5 mM CaCl₂ in a total volume of 60 μl for 10 mM at 37° C. The resuspended membranes are exposed to UV light as described above. The unbound nucleotides are removed by washing the membranes three times and then the washed membranes are resuspended for 1 h in 0.5 ml of 1% CHAPSO/HEPES, pH 7.4. The solubilized proteins are diluted 1:2 in HEPES buffer (final CHAPSO concentration=0.5%) and incubated overnight with X81 antibody for immunoprecipitation, as described previously (Fraering, P. C., et al. (2004) Biochemistry 43, 9774-9789, Fraering, P. C., et al. (2004) Biochemistry 43, 323-333). Samples are electrophoresed on 4-20% Tris-glycine gels and autoradiographed (BioMax MS films used with BioMax Transcreen HE (Eastman Kodak Co.)).

### Example 7

ATPase Assays—The following assay can be used to determine if the compounds of interest compete with ATP. [α-³²P]ATP (11.9 Ci/mmol) is purchased from Affinity Labeling Technology (Lexington, Ky.). The purified γ-secretase is prepared as described for the photoaffinity labeling experiments; 5 μCi of [α-³²P]ATP was added; the reactions are incubated at 37° C., and at the indicated time points aliquots are removed and reactions stopped by addition of 10% SDS. A total of 2 μl of each stopped reaction is analyzed by TLC on polyethyleneimine cellulose plastic sheets (Baker-Flex, Germany) with 0.75 M KH₂PO₄, pH 3.5, as the running buffer to separate ATP from ADP. To identify hydrolysis products, a reaction of [α-³²P]ATP can be incubated in the presence of 0.005 units of canine kidney phosphatase (Sigma). Samples are autoradiographed as described above.

### Example 8

Aβ(1-42) Cellular Assay—The following assay can be used to determine the extent to which the compounds of interest inhibit the cleavage of APP in vivo. Aβ ELISA is a commercial fluorometric kit from Biosource (Invitrogen 89344). Luciferase reporter HEK AP-GL-T16 cells are platedat 50,000 cells/well in 96 well plates in DMEM media containing 10% tetracyclin free BSA, 250 ug/ml zeocin, 200 ug/ml hygromycin, and 5 ug/ml blasticidin. Compounds are added 24 hr after plating and APP processing is induced simultaneously by addition of tetracycline. Following a 24 hr compound treatment, 50 ml of conditioned cell media is collected, mixed with ELISA diluent buffer containing 2 mM AEBSF and 12 mM o-phenanthroline, and immediately frozen at −80 degrees C. For the ELISA, the samples are brought to room temp and spun at 5000 rpm for 5 mM. 50 ml of sample are incubated in the ELISA plate with 50 ml detection antibody on a shaker at room temp for 3 hr. Wells are then washed 4 times with wash buffer and 100 ml of secondary antibody are added and incubated at room temp for 30 mM. Wells are again washed 4 times with wash buffer and 100 ml of fluorescent substrate solution are added. After 30 min incubation, fluorescent signals are determine on a Gemini reader at ex 460 nm and em 560 nm. The amount of Aβ levels in each sample is determined from a standard curve generated by known concentrations of Aβ peptide run simultaneously with the samples.

### Example 9

Determination of the effect of the number of cells on the luciferase signal—The following assay may be used to screen (e.g., in a high throughput screen) for candidate compounds that inhibit APP processing. The assay is performed with the AP-GL-T16 clone. Serially diluted cells, starting from 20,000 cells per well (80 μL), were added to 96-well plates. After 24 h of incubation, 20 μL of fresh media with/without 5 μg/mL of tetracycline (final concentration) is added. Two replicates for each condition are used. After 24 h of further incubation, 100 μL/well of luciferase substrate is added, and luminescence is checked on an LJL Analyst (Molecular Device).

### Example 10

EC50 determination with tetracycline—Cells are trypsinized using trypsin-EDTA (Invitrogen) and harvested by centrifugation at 1510 g. The pellet is then resuspended with DMEM-HZB. The density of cells is determined with a hematocytometer, and cells (500 cells/μL) are transferred at 40 μL/well into 384-well Nunc cell culture plates. Cells are incubated at 37° C. in a CO₂ incubator for 24 h. Serially diluted tetracycline is added to media starting from a 5-μg/mL concentration on a separate plate. For each concentration, 10 wells are used. For negative control, no tetracycline is added to media. On the second day, 10 μL/well of media with/without tetracycline is added. After an additional 48 h of incubation, the plates are brought to room temperature, and 50 μL of luciferase substrate is added. The luminescence is then read using an LJL Analyst (Molecular Device).

### Example 11

IC50 determination of a γ-secretase inhibitor—The following assay can be used to determine the concentration of a compound of the invention required to achieve 50% inhibition of γ-secretase activity. Serial 3-fold dilutions of compound E, a potent inhibitor of γ-secretase, starting at 3 μM final concentration, are prepared on a separate plate using media with tetracycline, and 10 μL of each is added to 384-well Nunc white plates containing cells (final concentration of tetracycline is 1 μg/mL). Ten replicates are used for each concentration, and the experiment is performed 3 times. The plates are further incubated for 48 h after tetracycline addition. After bringing the temperature down to room temperature, 50 μL of luciferase substrate/well is added and mixed, and luminescence is recorded with an LJL Analyst (Molecular Device).

### Example 12

MTS Assay—The following assay can be used to indicate the number of viable cells in proliferation and thereby evaluate the toxicity of a candidate compound. The MTS assay used is Promega's Cell Titer 96 Aqueous One Solution Cell Proliferation Assay. It is a colormetric assay that indicates the number of viable cells in proliferation by measuring the amount of MTS reduced to formazan by NADPH or NADH produced by metabolically active cells. After conditioned media is collected for the ELISA, MTS reagent is added to sample at a ratio of 20 ml reagent to 100 ml cell media. Samples are incubated for 1 hr at 37 degrees C. in a 5% CO₂ incubator. Then absorbance is recorded at 490 nm with a Gemini reader. Cell viability is assessed by determining the percent sample signal to untreated controls. All sample and control signals are adjusted to a background signal determined from cells lysed with 0.9% triton X.

### Example 13

Naphthyl Amine Alcohol Analogs and Assay Data—Compounds listed in Table 2 may be assayed as described herein (e.g., in Examples 3 and 4) and the specificity may be evaluated by comparing the relative inhibitions of C-100 Flag and N-100 Flag cleavage. The potency of the inhibitor may be evaluated with an Aβ1-40 ELISA assay (e.g., using an Aβ40 ELISA kit available from Invitrogen, Carlsbad, Calif.). In some embodiments specific inhibitors have a potency of at least 30% inhibition (e.g., or at least 40%, or at least 50%, or at least 60%, or at least 70%, or at least 80%, or at least 90%) and they inhibit C-100 Flag cleavage relative to N-100 cleavage (e.g., they don't inhibit N-100 cleavage or they enhance N-100 cleavage). The following table indicates the properties of certain compounds of the invention that were assayed as described herein. (INH=Inhibits)

TABLE 2

| | | | | γ-Secretase |
|---|---|---|---|---|
| | | | | Assay |
| | | γ-Secretase Assay | | ELISA |
| | | [μM] | | % Inhibition |
| Ex. | | C-100 | N-100 | [100 μM] |
| No. | Structure | Flag | Flag | Aβ1-40 |
| 1 | 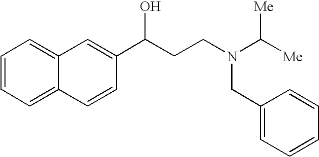 | Inhibits (Inh) | Enhances (Enh) | 16 |
| 2 | 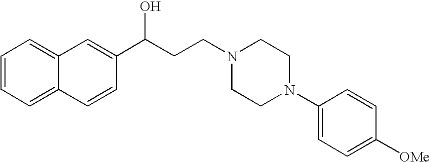 | No change (NC) | Not tested (NT) | 0 |
| 3 | 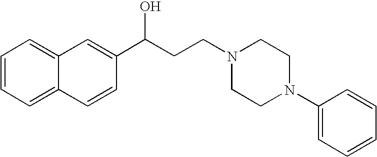 | NC | Enh | 0 |
| 4 | 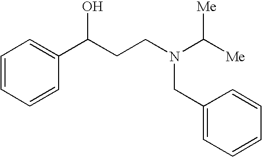 | NC | NT | 10 |
| 5 | 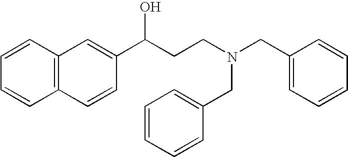 | NC | NT | 32 |
| 6 | 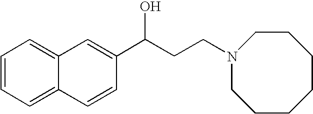 | NC | NT | 20 |
| 7 | 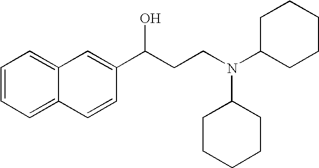 | NC | NT | 7 |
| 8 | 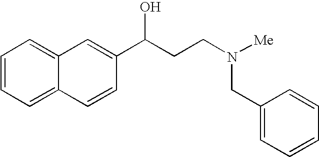 | NC | NT | 15 |
| 9 | 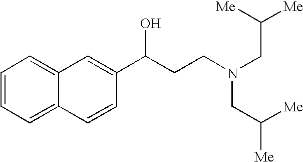 | NC | NT | 0 |
| 10 | 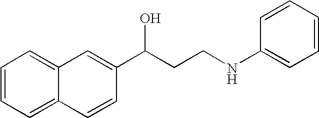 | Enh | NT | 0 |
| 11 | 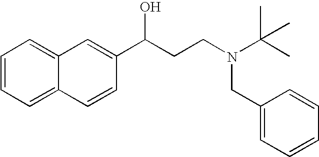 | NC | NT | 0 |
| 12 | 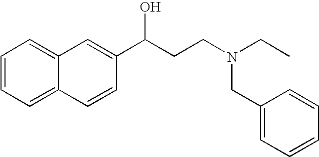 | Enh | NT | 0 |
| 13 | 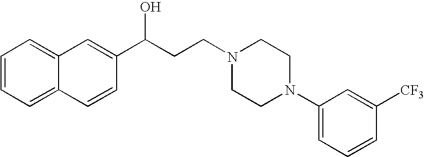 | Enh | Enh | 38 |
| 14 | 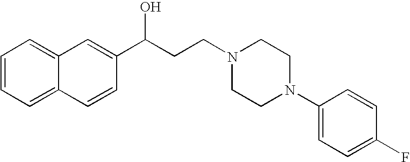 | Inh | Enh | 61 |
| 15 | 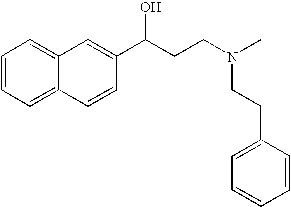 | NT | NT | 27 |
| 16 | 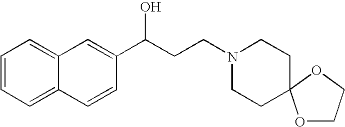 | NC | NT | 2 |
| 17 | 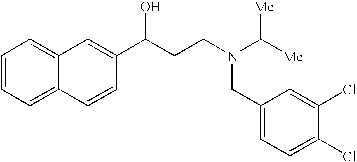 | Inh | Enh | 60 |
| 18 | 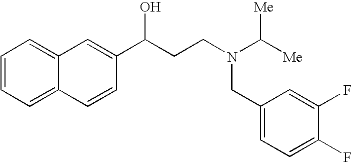 | Inh | Enh | 64 |
| 19 | 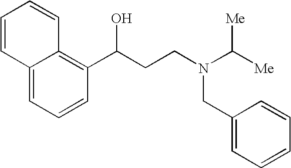 | Inh | Enh. | 46 |
| 20 | 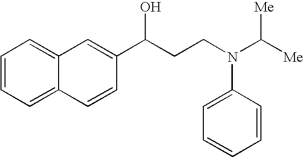 | Inh | Inh | 68 |
| 21 | 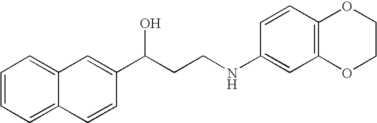 | NT | NT | 0 |
| 22 | 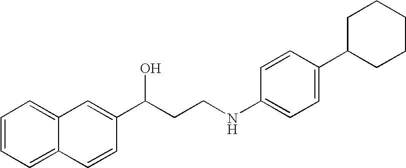 | NT | NT | 0 |
| 23 | 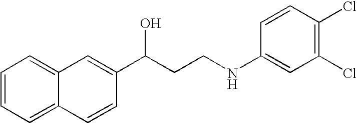 | NT | NT | 0 |
| 24 | 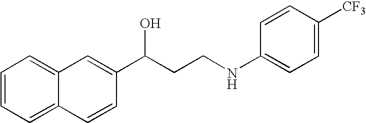 | NT | NT | 0 |
| 25 | 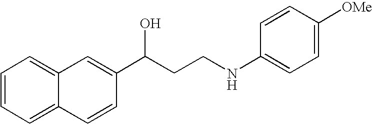 | NT | NT | 0 |
| 26 | 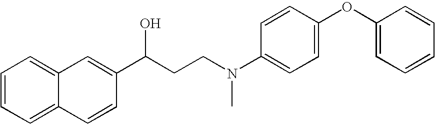 | NT | NT | 6 |
| 27 | 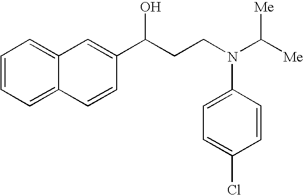 | NT | NT | 0 |
| 28 | 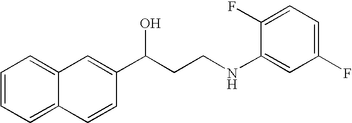 | NT | NT | 0 |
| 29 | 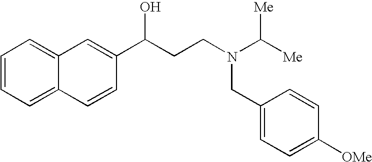 | Inh | NC | 55 |
| 30 | 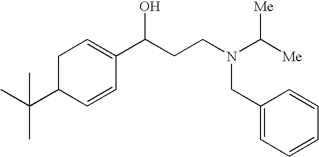 | Inh | Inh | 72 |
| 31 | 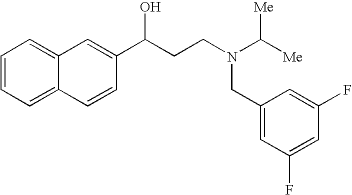 | Inh | Inh | 54 |
| 32 | 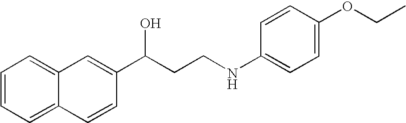 | Inh | Inh | 40 |
| 33 | 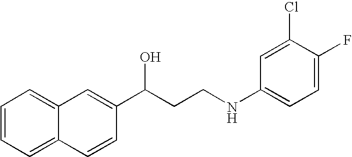 | Inh | Inh | 40 |
| 34 | 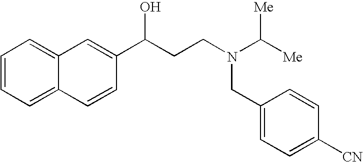 | NT | NT | 19 |
| 35 | 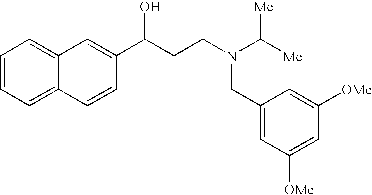 | NT | NT | 17 |
| 36 | 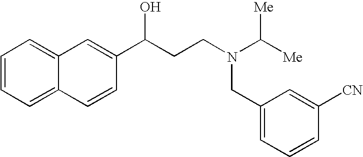 | NT | NT | 7 |
| 37 | 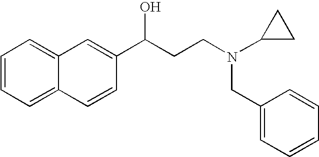 | NT | NT | 36 |
| 38 | 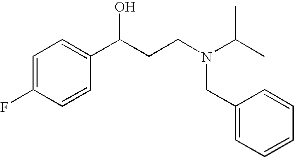 | NT | NT | 17 |
| 39 | 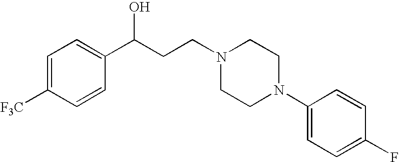 | NT | NT | 14 |
| 40 | 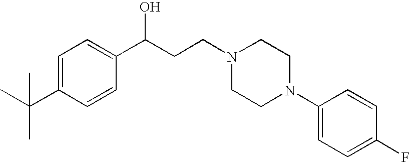 | NT | NC | 46 |
| 41 | 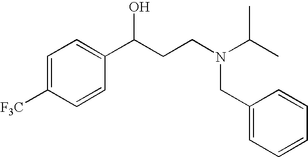 | NT | Enh | 27 |
| 42 | 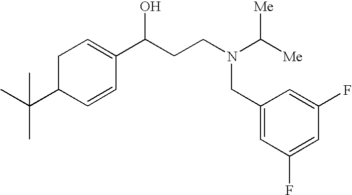 | NT | NC | 34 |
| 43 | 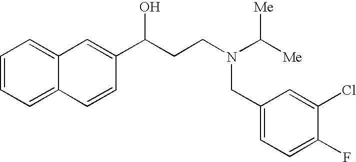 | NT | NT | 0 |
| 44 | 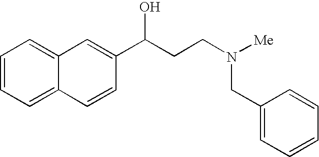 | NT | Enh | 56 |
| 45 | 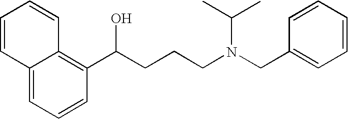 | NT | NC | 0 |
| 46 | 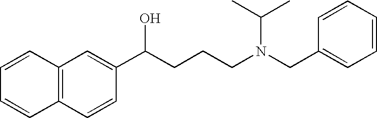 | NT | NT | 2 |
| 47 | 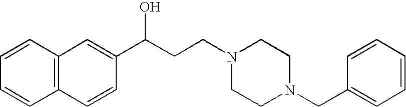 | NT | NT | 0 |
| 48 | 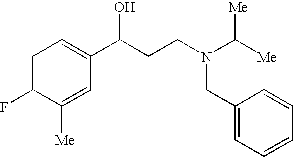 | NT | Inh | 4 |
| 49 | 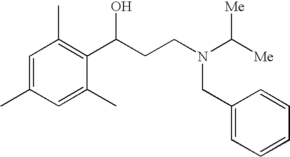 | NT | NT | 0 |
| 50 | 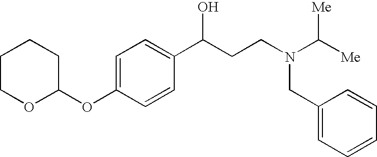 | NT | NT | 0 |
| 51 | 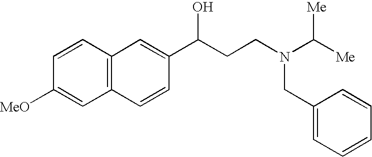 | NT | NT | 10 |
| 52 | 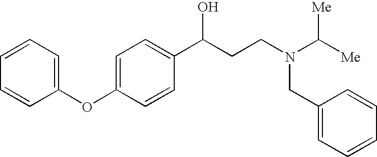 | NT | NC | 24 |
| 53 | 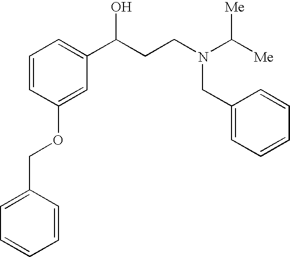 | NT | NC | 31 |
| 54 | 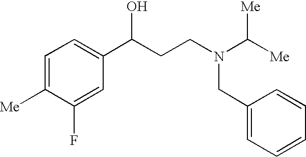 | NT | Inh | 29 |
| 55 | 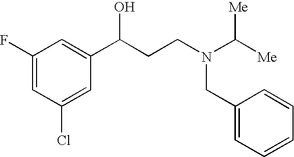 | NT | Inh | 30 |
| 56 | 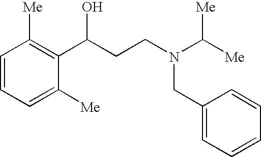 | NT | Inh | 20 |
| 57 | 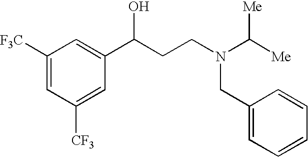 | NT | Inh | 47 |
| 58 | 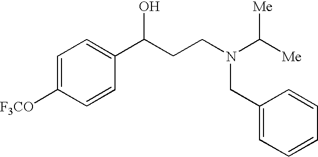 | NT | NT | 1 |
| 59 | 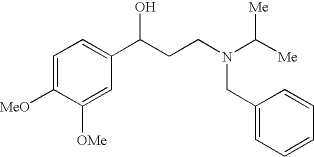 | NT | NT | 0 |
| 60 | 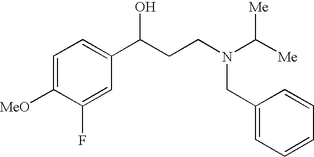 | NT | NT | 0 |
| 61 | 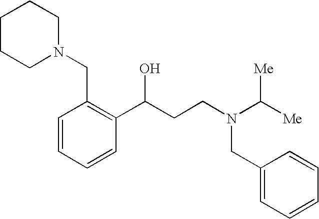 | NT | NT | 0 |
| 62 | 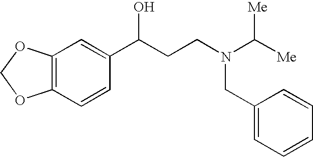 | NT | NT | 0 |
| 63 | 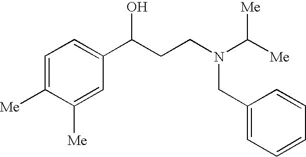 | NT | NT | 0 |
| 64 | 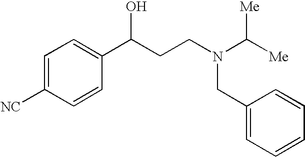 | NT | NC | 6 |
| 65 | 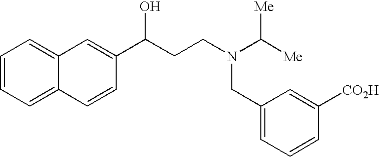 | NT | NT | NT |
| 66 | 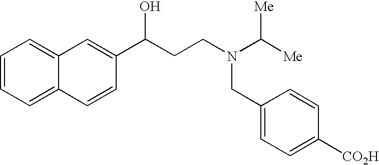 | NT | NT | 2 |
| 67 | 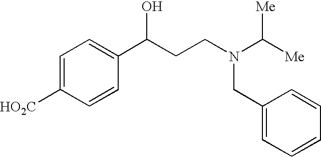 | NT | NC | 27 |
| 68 | 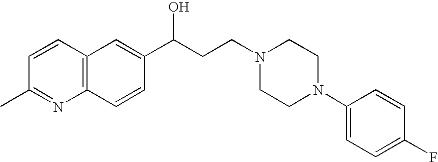 | NT | NT | NT |
| 69 | 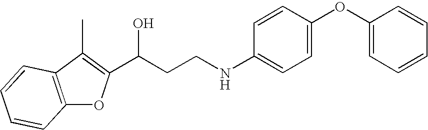 | NT | NT | NT |
| 70 | 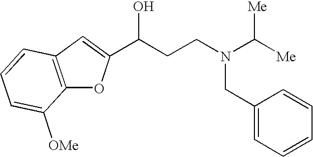 | NT | NT | 0 |
| 71 | 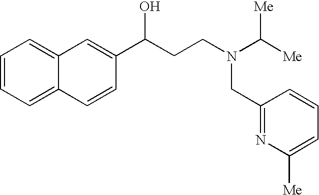 | NT | NT | NT |
| 72 | 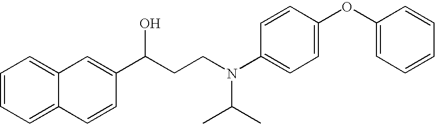 | NT | NT | NT |
| 73 | 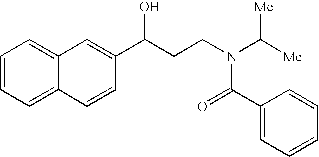 | NT | NT | NT |
| 74 | 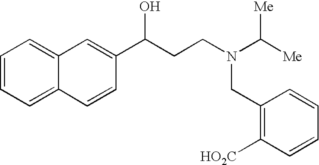 | NT | NT | NT |
| 75 | 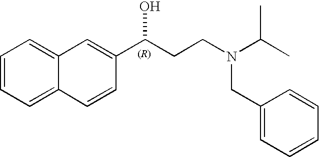 | NT | Inh | 0 |
| 76 | 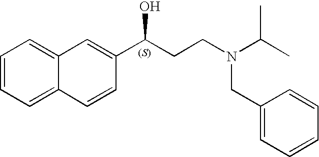 | NT | NC | 17 |
| 77 | 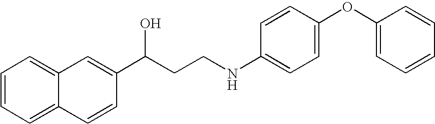 | Inh | NC | 48 |
| 78 | 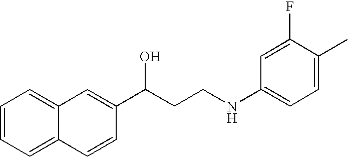 | Enh | NT | 0 |
| 79 | 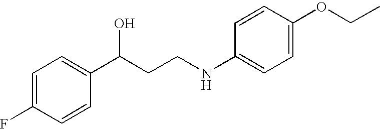 | Enh | NT | 0 |
| 80 | 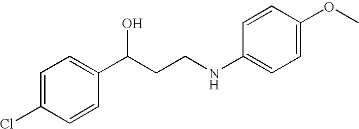 | Enh | NT | 0 |
| 81 | 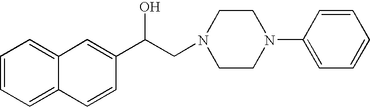 | NC | Enh | 0 |
| 82 | 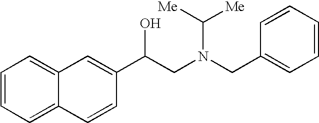 | Inh | Inh | 0 |
| 83 | 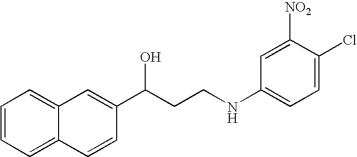 | Inh | Inh | 46 |
| 84 | 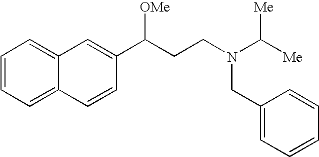 | Inh | NC | 51 |
| 85 | 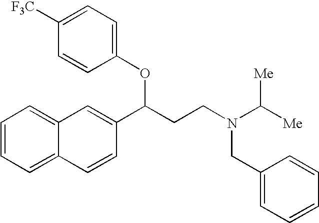 | NC | NC | 50 |
| 86 | 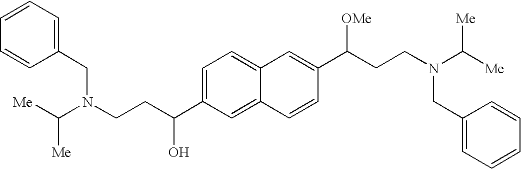 | NT | NT | 12 |
| 87 | 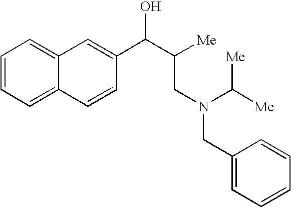 | NT | NC | 22 |
| 88 | 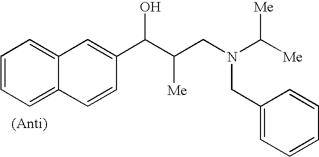 | Inh | NC | 66 |
| 89 | 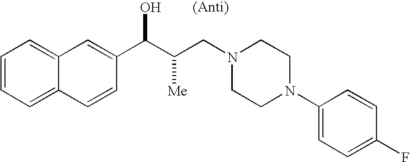 | NT | NT | 9 @ 50 μM |
| 90 | 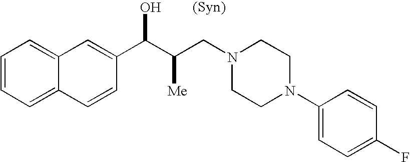 | NT | NT | 0 @ 50 μM |
| 91 | 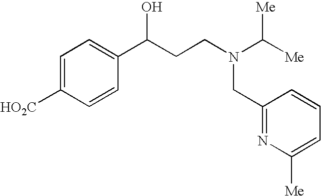 | NT | NT | NT |
| 92 | 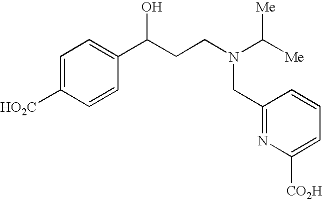 | NT | NT | NT |
| 93 | 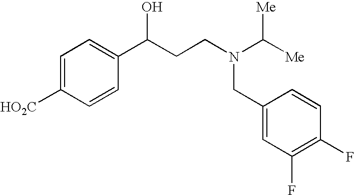 | NT | NT | NT |
| 94 | 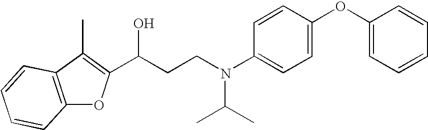 | NT | NT | NT |
| 95 | 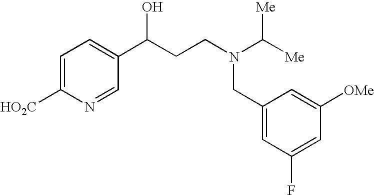 | NT | NT | NT |
| 96 | 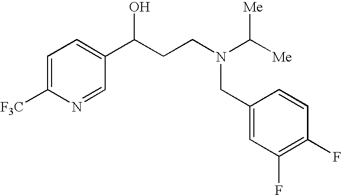 | NT | NT | NT |
| 97 | 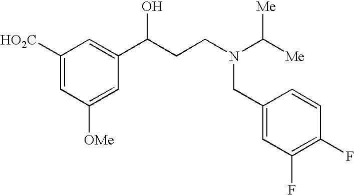 | NT | NT | NT |
| 98 | 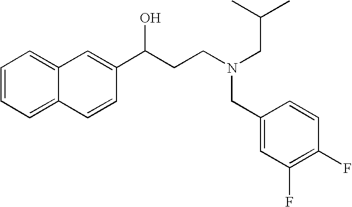 | NT | NT | NT |
| 99 | 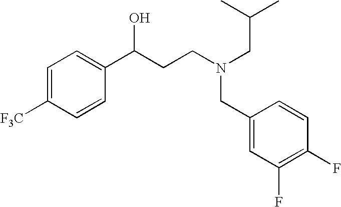 | NT | NT | NT |
| 100 | 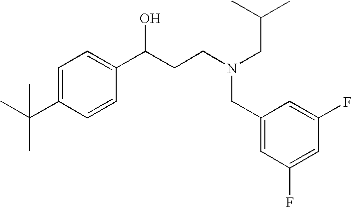 | NT | NT | NT |
| 101 | 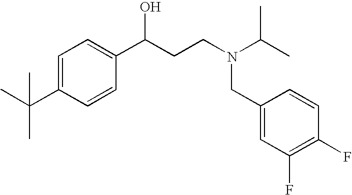 | NT | NT | NT |
| 102 | 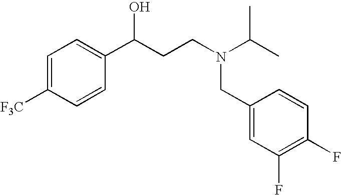 | NT | NT | NT |
| 103 | 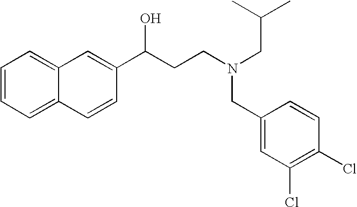 | NT | NT | NT |
| 104 | 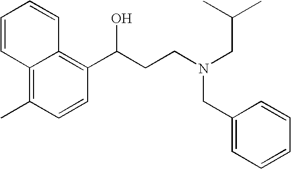 | NT | NT | NT |
| 105 | 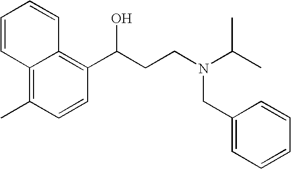 | NT | NT | NT |
| 106 | 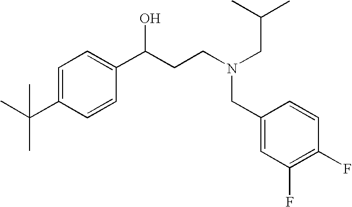 | NT | NT | NT |
| 107 | 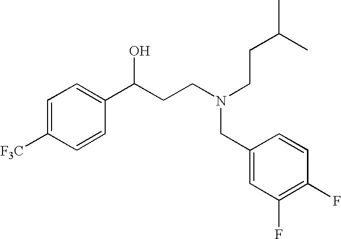 | NT | NT | NT |
| 108 | 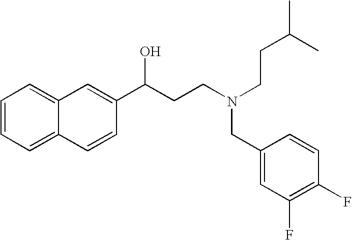 | NT | NT | NT |
| 109 | 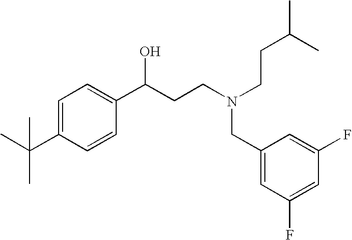 | NT | NT | NT |
| 110 | 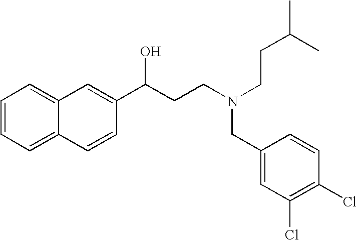 | NT | NT | NT |
| 111 | 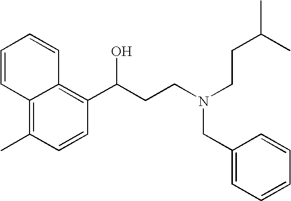 | NT | NT | NT |
| 112 | 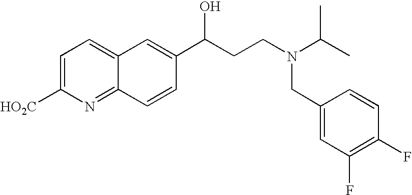 | NT | NT | NT |
| 113 | 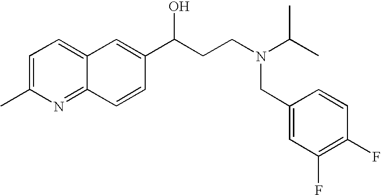 | NT | NT | NT |
| 114 | 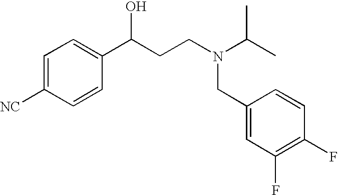 | NT | NT | NT |
| 115 | 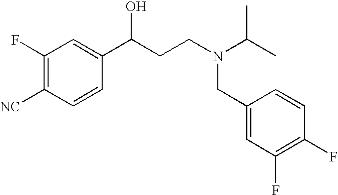 | NT | NT | NT |
| 116 | 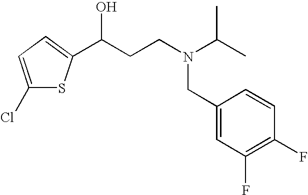 | NT | NT | NT |

In some embodiments, compounds were tested in in vitro assays at 100 μM. However, compounds of the invention (e.g., in this example, or described in the detailed description, may be used at any suitable concentration (e.g., from nm to mm, for example, from 1-5 μM, 5-50 μM, 50-200 μM, or at higher or lower concentrations). In some embodiments, a compound may be active at 100% inhibition at 50 μM. In some embodiments, compounds are not toxic (e.g., in an MTT assay at concentrations of 1-5 μM, 5-50 μM, 50-200 μM, or at higher or lower concentrations).

Having now described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A compound of formula 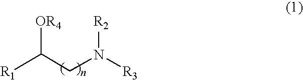
wherein:
R1 is selected from an aryl ring system;
R2 is alkyl or hydrogen;
R3 is either an alkyl group further substituted with alkyl or aryl groups or R3 is an aromatic ring system optionally substituted, or when taken together, R2 and R3 form an N,N′-substituted piperazine;
R4 is selected from hydrogen, alkyl, and aryl substituents; and,
n=1-4.

2. The compound of claim 1 wherein:
R1 is a bicyclic carbocyclic ring system.

3. The compound of claim 2 wherein:
R1 is an aromatic ring system.

4. The compound of claim 3 wherein:
R1 is selected from optionally substituted phenyl and naphthalene;
R4 is selected from hydrogen, and p-trifluoromethyl-substituted phenyl; and,
n=2.

5. The compound of claim 3 wherein:
R1 is optionally substituted naphthalene.

6. The compound of claim 5 wherein:
R4 is p-trifluoromethyl-substituted phenyl.

7. The compound of claim 6 wherein:
R4 is hydrogen.

8. The compound of claim 6 wherein:
R3 is an aromatic ring system, optionally substituted, or a benzyl substituent wherein its aromatic ring system is optionally substituted.

9. The compound of claim 8 wherein:
R2 is iso-propyl.

10. The compound of claim 9 wherein:
R3 is benzyl, optionally substituted.

11. The compound of claim 9 selected from the group consisting of:
3-(benzyl(isopropyl)amino)-1-(naphthalen-2-yl)propan-1-ol;
3-(4-(4-fluorophenyl)piperazin-1-yl)-1-(naphthalen-2-yl)propan-1-ol;
3-((3,4-difluorobenzyl)(isopropyl)amino)-1-(naphthalen-2-yl)propan-1-ol; and,
3-(benzyl(isopropyl)amino)-2-methyl-1-(naphthalen-2-yl)propan-1-ol.

12. The compound of claim 6 wherein:
R2 and R3 form a N,N′-substituted piperazine.

13. The compound of claim 12 wherein:
R2 and R3 form a N,N′ {p-trifluorophenyl-substituted phenyl}piperazine.

14. The compound of claim 2 wherein
R1 is an optionally substituted naphthalene;
X is O; and,
n=2.

15. The compound of claim 1, wherein the compound inhibits APP cleavage, relative to Notch cleavage, by γ-secretase.

16. A pharmaceutical composition comprising a compound of claim 1 or a salt thereof, and a pharmaceutical acceptable carrier.

17. The pharmaceutical composition of claim 16, further comprising a non-γ-secretase inhibitor compound.

18. The pharmaceutical composition of claim 16, further comprising an additional γ-secretase inhibitor compound.

19. A method of treating a subject having a condition associated with γ-secretase activity, or at risk of developing a condition associated with γ-secretase activity, the method comprising administering to a subject in need thereof a therapeutically effective amount of a compound of claim 1.

20. A method of treating a subject having Alzheimer's disease, or at risk of developing Alzheimer's disease, the method comprising administering to a subject in need thereof a therapeutically effective amount of a compound of claim 1.

21. A method of inhibiting APP cleavage, the method comprising contacting γ-secretase with a compound of claim 1.

22. The method of claim 19, wherein the compound does not inhibit Notch processing or degradation.

23. A kit comprising a compound of claim 1.
